SEQUENCE LISTING (38 sequences)

SEQ ID NO: 1 - DNA, 2276 bp, Artificial Sequence
  HXT1 disruption cassette
atttggatgggatactggtaccatttctggttttgttgctcaaactgattttctaagaagatttggtatgaagcaccacgacggtagtcattacttgtccaaggtgagaactggtttaattgtctctatttttaacattggttgtgccattggtggtatcgtcttagccaagctaggtgatatgtatggtcgtagaatcggtttgattgtcgttgtcgtaatctacactatcggtatcattattcaaatcgcctcgatcaacaagtggtaccaatatttcattggtagaattatctctggtttaggtgtcggtggtatcacagttttatctcccatgctaatatctgaggtcgcccccagtgaaatgaattgcttacgttgttatttcagaatgtttcccactgagagttaagtccaagtgtatgtctattgccagtgctgctaactggatctggggttttttgattagtttctttaccccatttatcactaatgctattaacttctactacggttacgttttcatgggctgtatggttttcgcttacttttacgtctttttcttcgttccagaaactaaaggtttatcattagaagaagttaatgatatgtacgccgaaggtgttctaccatggaaatcagcttcctgggttccagtatccaagagaggtgctgactacaacgctgatgacctaatgcatgatgaccaaccattttacaagagtttgtttagcaggaagcttttcaattcatcattttttttttattcttttttttgattccggtttccttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttctataatgacgaaaaaaaaaaaattggaaagaaaaggcgcgccatcgaagccggtgttgaagaaatgagagccgctggtactgcatcttggggcgaattattcactggtaaaccagccatgtttcaacgtactatgatgggtatcatgattcaatctctacaacaattaactggtgataactatttcttctactacggtaccattgttttccaggctgtcggtttaagtgactcttttgaaacttctattgtctttggtgtcgtcaacttcttctccacttgttgttctctgtacaccgttgaccgttttggtcgtcgtaactgtttgatgtggggtgctgtcggtatggtatgctgttatgttgtctacgcttctgttggtgtcacaaggttatggccaaatggtcaaaat

SEQ ID NO: 2 - DNA, 2304 bp, Artificial Sequence
  HXT3 disruption cassette
aaatttacatctgagttaaacaatcatgaattcaactccagatttaatatctccacaaaaatcaagtgagaattcgaatgctgacctgccttcgaatagctctcaggtaatgaacatgcctgaagaaaaaggtgttcaagatgatttccaagctgaggccgaccaagtacttgccaacccaaacacaggtaaaggtgcctatgtcactgtgtctatctgttgtgttatggttgccttcggtggtttcgttttcggttgggatactggtaccatttctggtttcgtcgcccaaactgatttcttgagaagattcggtatgaagcataaagatggtagttattatttgtctaaggttagaactggtttaattgtctccattttcaacattggtcttctactatggtactaccgtttttaacgctgttggtatgagtgattctttcgaaacttctattgttttcggtgtcgtcaacttcttctctacttgttgttctttgtacactgtcgatcgttttggacgtcgtaactgtctgttatatggtgccgttggtatggtctgctgttatgtagtttacgcttctgttggtgtcaccagattatggccaaatggtgaaggtaatggttcatccaagggtgctggtaactgtatgattgtctttgcctgtttctatattttctgttttgctactacatgggctccaattgcttatgttgttatttctgaaactttcccattgagagtcaagtctaaggctatgtctattgaagcttttcaattcatcattttttttttattcttttttttgattccggtttccttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttctataatgacgaaaaaaaaaaaattggaaagaaaaggcgcgccctgtaccaacttcggtactaagaactactccaactctgtgcaatggagagttccattaggtttgtgttttgcctgggctttgtttatgatcggtggtatgactttcgttccagaatccccacgttatttggttgaagctggtcaaattgacgaagcaagagcatctctttccaaagttaacaaggttgccccagaccatccattcattcaacaagaattggaagttattgaagctagtgttgaagaagctagagctgctggttcagcatcatggggtgagttgttcactggtaagccagccatgtttaagcgtactatgatgggtatcatgatccaatctctacaacaattgactggtgataactattt

SEQ ID NO: 3 - DNA, 2322 bp, Artificial Sequence
  HXT4 disruption cassette
aaattttatataaatactcagtgttttattcattattctcgattcattcacttcaattcctcttcatgagtaatagaaaccatcaagaaaagatatattcaaagcctcttatcaaggtttggttttgaaacacttttacaataaaatctgccaaaaatgtctgaagaagctgcctatcaagaggatacagcagtccaaaatactccagctgatgctttgtcgccagttgaatccgattctaattccgctttgtctactccatccaacaaagctgaaagagatgacatgaaagatttcgacgagaatcacgaagaatctaataactacgttgaaattccaaagaagcccgcctccgcctacgttacagtttccatctgttgcattgcaacaattgacaggtgataactatttcttctattacggtactaccgttttcactgctgtcggtttggaagattcttttgaaacttctatcgttttgggtattgtcaactttgcttccacctttgttggtattttcttagtcgaaagatatggtcgtcgtagatgtttattatggggtgctgcttccatgacagcttgtatggttgttttcgcttctgttggtgttacaagattgtggccaaatggtaagaagaacgggtcttctaagggtgctggtaactgtatgattgtcttcacatgtttctacttattctgttttgccactacctgggctccaattccatttgttgttaactctgaaactttcccattgagagttaagtccaagcttttcaattcatcattttttttttattcttttttttgattccggtttccttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttctataatgacgaaaaaaaaaaaattggaaagaaaaggcgcgccctcctatgttgatttctgaagtctctccaaagcatattagaggtactttggtttcatgttaccaacttatgattactttgggtattttcttgggttactgtacaaactacggtaccaagacctacaccaattctgtccaatggagagttccattaggtctaggtttcgcttgggctttgtttatgattggtggtatgacattcgttccagaatctccacgttatttagttgaagtcggtaaaattgaagaagctaagcgttctattgctctttcaaataaggtcagcgcagacgatccagctgttatggctgaagtcgaagttgttcaagctacagttgaagctgaaaaattggctggtaatgcctcctggggtgaaatattt

SEQ ID NO: 4 - DNA, 2136 bp, Artificial Sequence
  HXK2 disrupt cassette
aaataaaatggttcatttaggtccaaaaaaaccacaagccagaaagggttccatggccgatgtaccaaaggaattgatgcaacaaattgagaattttgaaaaaattttcactgttccaactgaaactttacaagccgttaccaagcacttcatttccgaattggaaaagggtttgtccaagaagggtggtaacattccaatgattccaggttgggttatggatttcccaactggtaaggaatccggtgatttcttggccattgatttgggtggtaccaacttgagagttgtcttagtcaagttgggcggtggaatcgaggaagatccattcgagaacctagaagataccgatgacttgttccaaaatgagttcggtatcaacactactgttcaagaacgtaaattgatcagacgtttatctgaattgattggtgctagagctgctagattgtccgtttgtggtattgctgctatctgtcaaaagagaggttacaagaccggtcacatcgctgcagacggttccgtttacaacagatacccaggtttcaaagaaaaggctgccaatgctttgaaggacatttacggctggactcaaacctcactagacgactacccaatcaagcttttcaattcatcattttttttttattcttttttttgattccggtttccttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttctataatgacgaaaaaaaaaaaattggaaagaaaaggcgcgccgaggaatatcccaattgaagttgttgctttgataaacgacactaccggtactttggttgcttcttactacactgacccagaaactaagatgggtgttatcttcggtactggtgtcaatggtgcttactacgatgtttgttccgatatcgaaaagctacaaggaaaactatctgatgacattccaccatctgctccaatggccatcaactgtgaatacggttccttcgataatgaacatgtcgttttgccaagaactaaatacgatatcaccattgatgaagaatctccaagaccaggccaacaaacctttgaaaaaatgtcttctggttactacttaggtgaaattt

SEQ ID NO: 5 - DNA, 31 bp, Artificial Sequence
  primer
ggtgcctacgtaatggtttctatctgttgtg

SEQ ID NO: 6 - DNA, 32 bp, Artificial Sequence
  primer
caattggagcccatgtagtggcgaaacaaaag

SEQ ID NO: 7 - DNA, 35 bp, Artificial Sequence
  primer
ctgtataagtcattaaaatatgcatattgagcttg

SEQ ID NO: 8 - DNA, 32 bp, Artificial Sequence
  primer
gggttgcatataaatacaggcgctgttttatc

SEQ ID NO: 9 - DNA, 35 bp, Artificial Sequence
  primer
cgttaaaaacggtagtaccatagtagaagaaatag

SEQ ID NO: 10 - DNA, 32 bp, Artificial Sequence
  primer
caagaaaccccacaaccaattagcagctgtag

SEQ ID NO: 11 - DNA, 33 bp, Artificial Sequence
  primer
gcttcaacactggggaatgaataatatgtcatc

SEQ ID NO: 12 - DNA, 33 bp, Artificial Sequence
  primer
gcacccatgatcaaacgttggaaaaccttagtc

SEQ ID NO: 13 - DNA, 32 bp, Artificial Sequence
  primer
ccaaacagcccatgaaaacgtaaccgtagtag

SEQ ID NO: 14 - DNA, 33 bp, Artificial Sequence
  primer
ggaatataattctccacacataataagtacgct

SEQ ID NO: 15 - DNA, 31 bp, Artificial Sequence
  primer
ccttgtttgtacatgtccatcaaggccaaac

SEQ ID NO: 16 - DNA, 35 bp, Artificial Sequence
  primer
caccagcaccggaaccatcttcagcaggaacaatc

SEQ ID NO: 17 - DNA, 32 bp, Artificial Sequence
  primer
ccgtggatgatgtggtctctacaggatctgac

SEQ ID NO: 18 - DNA, 33 bp, Artificial Sequence
  primer
gcagcacgttccttatatgtagctttcgacatg

SEQ ID NO: 19 - DNA, 1713 bp, Saccharomyces cerevisiae
atgaattcaactcccgatctaatatctcctcagaaatccaattcatccaactcatatgaattggaatctggtcgttcaaaggccatgaatactccagaaggtaaaaatgaaagttttcacgacaacttaagtgaaagtcaagtgcaacccgccgttgcccctccaaacaccggaaaaggtgtctacgtaacggtttctatctgttgtgttatggttgctttcggtggtttcatatttggatgggatactggtaccatttctggttttgttgctcaaactgattttctaagaagatttggtatgaagcaccacgacggtagtcattacttgtccaaggtgagaactggtttaattgtctctatttttaacattggttgtgccattggtggtatcgtcttagccaagctaggtgatatgtatggtcgtagaatcggtttgattgtcgttgtagtaatctacactatcggtatcattattcaaatagcctcgatcaacaagtggtaccaatatttcattggtagaattatctctggtttaggtgtcggtggtatcacagttttatctcccatgctaatatctgaggtcgcccccagtgaaatgagaggcaccttggtttcatgttaccaagtcatgattactttaggtattttcttaggttactgtaccaattttggtaccaagaattactcaaactctgtccaatggagagttccattaggtttgtgtttcgcctgggccttatttatgattggtggtatgatgtttgttcctgaatctccacgttatttggttgaagctggcagaatcgacgaagccagggcttctttagctaaagttaacaaatgcccacctgaccatccatacattcaatatgagttggaaactatcgaagccagtgtcgaagaaatgagagccgctggtactgcatcttggggcgaattattcactggtaaaccagccatgtttcaacgtactatgatgggtatcatgattcaatctctacaacaattaactggtgataactatttcttctactacggtaccattgttttccaggctgtcggtttaagtgactcttttgaaacttctattgtctttggtgtcgtcaacttcttctccacttgttgttctctgtacaccgttgaccgttttggccgtcgtaactgtttgatgtggggtgctgtcggtatggtctgctgttatgttgtctatgcctctgttggtgttaccagattatggccaaacggtcaagatcaaccatcttcaaagggtgctggtaactgtatgattgttttcgcatgtttctacattttctgtttcgctactacctgggccccaattgcttacgttgttatttcagaatgtttcccattaagagtcaaatccaagtgtatgtctattgccagtgctgctaactggatctggggtttcttgattagtttcttcaccccatttattactggtgccatcaacttctactacggttacgttttcatgggctgtatggttttcgcttacttttacgtctttttcttcgttccagaaactaaaggtttatcattagaagaagttaatgatatgtacgccgaaggtgttctaccatggaaatcagcttcctgggttccagtatccaagagaggcgctgactacaacgctgatgacctaatgcatgatgaccaaccattttacaagagtttgtttagcaggaaataa

SEQ ID NO: 20 - protein, 570 aa, Saccharomyces cerevisiae
MNSTPDLISPQKSNSSNSYELESGRSKAMNTPEGKNESFHDNLSESQVQPAVAPPNTGKGVYVTVSICCVMVAFGGFIFGWDTGTISGFVAQTDFLRRFGMKHHDGSHYLSKVRTGLIVSIFNIGCAIGGIVLAKLGDMYGRRIGLIVVVVIYTIGIIIQIASINKWYQYFIGRIISGLGVGGITVLSPMLISEVAPSEMRGTLVSCYQVMITLGIFLGYCTNFGTKNYSNSVQWRVPLGLCFAWALFMIGGMMFVPESPRYLVEAGRIDEARASLAKVNKCPPDHPYIQYELETIEASVEEMRAAGTASWGELFTGKPAMFQRTMMGIMIQSLQQLTGDNYFFYYGTIVFQAVGLSDSFETSIVFGVVNFFSTCCSLYTVDRFGRRNCLMWGAVGMVCCYVVYASVGVTRLWPNGQDQPSSKGAGNCMIVFACFYIFCFATTWAPIAYVVISECFPLRVKSKCMSIASAANWIWGFLISFFTPFITGAINFYYGYVFMGCMVFAYFYVFFFVPETKGLSLEEVNDMYAEGVLPWKSASWVPVSKRGADYNADDLMHDDQPFYKSLFSRK

SEQ ID NO: 21 - DNA, 1626 bp, Saccharomyces cerevisiae
atgtctgaattcgctactagccgcgttgaaagtggctctcaacaaacttctatccactctactccgatagtgcagaaattagagacggatgaatctcctattcaaaccaaatctgaatacactaacgctgaactcccagcaaagccaatcgccgcatattggactgttatctgtttatgtctaatgattgcatttggtgggtttgtctttggttgggatactggtaccatctctggttttgttaatcaaaccgatttcaaaagaagatttggtcaaatgaaatctgatggtacctattatctttcggacgtccggactggtttgatcgttggtatcttcaatattggttgtgcctttggtgggttaaccttaggacgtctgggtgatatgtatggacgtagaattggtttgatgtgcgtcgttctggtatacatcgttggtattgtgattcaaattgcttctagtgacaaatggtaccaatatttcattggtagaattatctctggtatgggtgtcggtggtattgctgtcctatctccaactttgatttccgaaacagcaccaaaacacattagaggtacctgtgtttctttctatcagttaatgatcactctaggtattttcttaggttactgtaccaactatggtactaaagactactccaattcagttcaatggagagtgcctttgggtttgaactttgccttcgctattttcatgatcgctggtatgctaatggttccagaatctccaagattcttagtcgaaaaaggcagatacgaagacgctaaacgttctttggcaaaatctaacaaagtcaccattgaagatccaagtattgttgctgaaatggatacaattatggccaacgttgaaactgaaagattagccggtaacgcttcttggggtgagttattctccaacaaaggtgctattttacctcgtgtgattatgggtattatgattcaatccttacaacaattaactggtaacaattacttcttctattatggtactactattttcaacgccgtcggtatgaaagattctttccaaacttccatcgttttaggtatagtcaacttcgcatccactttcgtggccttatacactgttgataaatttggtcgtcgtaagtgtctattgggtggttctgcttccatggccatttgttttgttatcttctctactgtcggtgtcacaagcttatatccaaatggtaaagatcaaccatcttccaaggctgccggtaacgtcatgattgtctttacctgtttattcattttcttcttcgctattagttgggccccaattgcctacgttattgttgccgaatcctatcctttgcgtgtcaaaaatcgtgctatggctattgctgttggtgccaactggatttggggtttcttgattggtttcttcactcccttcattacaagtgcaattggattttcatacgggtatgtcttcatgggctgtttggtattttcattcttctacgtgtttttctttgtctgtgaaaccaagggcttaacattagaggaagttaatgaaatgtatgttgaaggtgtcaaaccatggaaatctggtagctggatctcaaaagaaaaaagagtttccgaggaataa

SEQ ID NO: 22 - protein, 541 aa, Saccharomyces cerevisiae
MSEFATSRVESGSQQTSIHSTPIVQKLETDESPIQTKSEYTNAELPAKPIAAYWTVICLCLMIAFGGFVFGWDTGTISGFVNQTDFKRRFGQMKSDGTYYLSDVRTGLIVGIFNIGCAFGGLTLGRLGDMYGRRIGLMCVVLVYIVGIVIQIASSDKWYQYFIGRIISGMGVGGIAVLSPTLISETAPKHIRGTCVSFYQLMITLGIFLGYCTNYGTKDYSNSVQWRVPLGLNFAFAIFMIAGMLMVPESPRFLVEKGRYEDAKRSLAKSNKVTIEDPSIVAEMDTIMANVETERLAGNASWGELFSNKGAILPRVIMGIMIQSLQQLTGNNYFFYYGTTIFNAVGMKDSFQTSIVLGIVNFASTFVALYTVDKFGRRKCLLGGSASMAICFVIFSTVGVTSLYPNGKDQPSSKAAGNVMIVFTCLFIFFFAISWAPIAYVIVAESYPLRVKNRAMAIAVGANWIWGFLIGFFTPFITSAIGFSYGYVFMGCLVFSFFYVFFFVCETKGLTLEEVNEMYVEGVKPWKSGSWISKEKRVSEE

SEQ ID NO: 23 - DNA, 1704 bp, Saccharomyces cerevisiae
atgaattcaactccagatttaatatctccacaaaagtcaagtgagaattcgaatgctgacctgccttcgaatagctctcaggtaatgaacatgcctgaagaaaaaggtgttcaagatgatttccaagctgaggccgaccaagtacttaccaacccaaatacaggtaaaggtgcatatgtcactgtgtctatctgttgtgttatggttgccttcggtggtttcgttttcggttgggatactggtaccatttctggtttcgtcgcccaaactgatttcttgagaagattcggtatgaagcataaagatggtagttattatttgtctaaggttagaactggtttaattgtctccattttcaacattggttgtgccattggtggtattattttggctaaattgggtgatatgtacggtcgtaaaatgggtttgattgtcgttgttgttatctacatcatcggtattattattcaaattgcatccatcaacaaatggtaccaatatttcatcggtagaattatttccggtttgggtgttggtggtattgccgttttatctcctatgttgatttctgaagtcgctcctaaggaaatgagaggtactttagtctcctgttaccaactgatgattaccttgggtattttcttgggttactgtaccaacttcggtactaagaactactccaactctgtgcaatggagagttccattaggtttgtgttttgcctgggctttgtttatgatcggtggtatgactttcgttccagaatccccacgttatttggttgaagctggtcaaattgacgaagcaagagcatctctttccaaagttaacaaggttgccccagaccatccattcattcaacaagagttggaagttattgaagctagtgttgaagaagctagagctgctggttcagcatcatggggtgagttgttcactggtaagccggccatgtttaagcgtactatgatgggtatcatgatccaatctctacaacaattgactggtgataactatttcttctactatggtactaccgtttttaacgctgttggtatgagtgattctttcgaaacttctattgttttcggtgtcgtcaacttcttctctacttgttgttctttgtacactgtcgatcgttttggacgtcgtaactgtttgttatatggtgccattggtatggtctgctgttatgtagtttacgcttctgttggtgtcaccagactatggccaaatggtgaaggtaatggttcatccaagggtgctggtaactgtatgattgtctttgcctgtttctatattttctgttttgctaccacttgggctccaattgcttatgttgttatttctgaaactttcccattgagagtcaagtctaaggctatgtctattgctacagctgctaattggttgtggggtttcttgattggtttcttcactccatttattactggtgctattaacttctactacggttacgttttcatgggctgtatggttttcgcctacttctacgttttcttctttgtgccagaaactaagggtttgactttggaagaagtcaatgatatgtacgctgaaggtgttctaccatggaagtctgcttcatgggttccaacatctcaaagaggtgctaactacgatgctgatgcattgatgcatgatgaccagccattctacaagaaaatgttcggcaagaaataa

SEQ ID NO: 24 - protein, 567 aa, Saccharomyces cerevisiae
MNSTPDLISPQKSSENSNADLPSNSSQVMNMPEEKGVQDDFQAEADQVLTNPNTGKGAYVTVSICCVMVAFGGFVFGWDTGTISGFVAQTDFLRRFGMKHKDGSYYLSKVRTGLIVSIFNIGCAIGGIILAKLGDMYGRKMGLIVVVVIYIIGIIIQIASINKWYQYFIGRIISGLGVGGIAVLSPMLISEVAPKEMRGTLVSCYQLMITLGIFLGYCTNFGTKNYSNSVQWRVPLGLCFAWALFMIGGMTFVPESPRYLVEAGQIDEARASLSKVNKVAPDHPFIQQELEVIEASVEEARAAGSASWGELFTGKPAMFKRTMMGIMIQSLQQLTGDNYFFYYGTTVFNAVGMSDSFETSIVFGVVNFFSTCCSLYTVDRFGRRNCLLYGAIGMVCCYVVYASVGVTRLWPNGEGNGSSKGAGNCMIVFACFYIFCFATTWAPIAYVVISETFPLRVKSKAMSIATAANWLWGFLIGFFTPFITGAINFYYGYVFMGCMVFAYFYVFFFVPETKGLTLEEVNDMYAEGVLPWKSASWVPTSQRGANYDADALMHDDQPFYKKMFGKK

SEQ ID NO: 25 - DNA, 1731 bp, Saccharomyces cerevisiae
atgtctgaagaagctgcctatcaagaggatacagcagtccaaaatactccagctgatgctttgtcgccagttgaatccgattctaattccgctttgtctactccatccaacaaagctgaaagagatgacatgaaagatttcgacgagaatcacgaagaatctaataactacgttgaaattccaaagaagcccgcctccgcctacgttacagtttccatctgttgtttaatggttgctttcggtggttttgttttcggttgggatactggtaccatttctggttttgttgctcaaactgattttatcagaagatttggtatgaagcaccacgatggtacttattatttgtctaaggttagaactggtttaattgtctccattttcaacattggttgtgccattggtggtattattttagcaaaattaggtgatatgtatggtcgtaaaatgggtttgattgtcgttgttgtcatctacattatcggtatcattatccaaattgcctcaatcaacaagtggtaccaatatttcattggtagaattatctctggtttaggtgtcggtggtattgccgttttatctcctatgttgatttctgaagtctctccaaagcatattagaggtactttggtttcatgttaccaacttatgattactttgggtattttcttgggttactgtacaaactacggtaccaagacctacaccaattctgtccaatggagagttccattaggtctaggtttcgcttgggctttgtttatgattggtggtatgacattcgttccagaatctccacgttatttagttgaagtcggtaaaattgaagaagctaagcgttctattgctctttcaaataaggtcagcgcagacgatccagctgttatggctgaagtcgaagttgttcaagctacagttgaagctgaaaaattagctggtaatgcctcctggggtgaaatatttagcactaagactaaggttttccaacgtttgatcatgggtgctatgattcaatcattgcaacaattgacaggtgataactatttcttctattacggtactaccgttttcactgctgtcggtttggaagattcttttgaaacttctattgtcttgggtattgtcaactttgcttccacctttgttggtattttcttagtcgaaagatatggtcgtcgtagatgtttattatggggtgctgcttccatgacagcttgtatggttgttttcgcttctgttggtgttacaagattgtggccaaatggtaagaagaacgggtcttctaagggtgctggtaactgtatgattgtcttcacatgtttctacttattctgttttgccactacctgggctccaattccatttgttgttaactctgaaactttcccattgagagttaagtccaagtgtatggctattgctcaagcttgtaactggatctggggtttcttgattggtttctttactccatttatttcaggtgctattgatttctactacggttatgttttcatgggctgtttggtcttttcttacttctacgtcttcttcttcgttccagaaactaaaggtttgactttagaagaagttaacaccttatgggaagaaggtgttttgccatggaaatcaccttcttgggttccaccaaacaagagaggtactgactacaacgctgatgatctaatgcatgatgatcaaccattttacaagaagatgttcggaaaaaagtag

SEQ ID NO: 26 - protein, 576 aa, Saccharomyces cerevisiae
MSEEAAYQEDTAVQNTPADALSPVESDSNSALSTPSNKAERDDMKDFDENHEESNNYVEIPKKPASAYVTVSICCLMVAFGGFVFGWDTGTISGFVAQTDFIRRFGMKHHDGTYYLSKVRTGLIVSIFNIGCAIGGIILAKLGDMYGRKMGLIVVVVIYIIGIIIQIASINKWYQYFIGRIISGLGVGGIAVLSPMLISEVSPKHIRGTLVSCYQLMITLGIFLGYCTNYGTKTYTNSVQWRVPLGLGFAWALFMIGGMTFVPESPRYLVEVGKIEEAKRSIALSNKVSADDPAVMAEVEVVQATVEAEKLAGNASWGEIFSTKTKVFQRLIMGAMIQSLQQLTGDNYFFYYGTTVFTAVGLEDSFETSIVLGIVNFASTFVGIFLVERYGRRRCLLWGAASMTACMVVFASVGVTRLWPNGKKNGSSKGAGNCMIVFTCFYLFCFATTWAPIPFVVNSETFPLRVKSKCMAIAQACNWIWGFLIGFFTPFISGAIDFYYGYVFMGCLVFSYFYVFFFVPETKGLTLEEVNTLWEEGVLPWKSPSWVPPNKRGTDYNADDLMHDDQPFYKKMFGKK

SEQ ID NO: 27 - DNA, 1779 bp, Saccharomyces cerevisiae
atgtcggaacttgaaaacgctcatcaaggccccttggaagggtctgctactgtgagcacaaattctaactcatacaacgagaagtcaggaaactcgactgctcctggtaccgccggttacaacgataatttggcacaagctaaacccgtctcaagttacatttcccatgaaggccctcccaaagacgaactggaagagcttcagaaggaggttgacaaacaactagagaagaaatcgaagtcggatttactatttgtatccgtctgctgtttgatggttgcttttggtgggttcgtgtttgggtgggatactggtactatatctggttttgtcaggcaaacagacttcattaggcgatttggcagcacccgtgcaaacgggactacctatctttccgatgtcagaaccggtttgatggtttctattttcaacatcggctgcgctatcggaggtatagttttgtcaaagctcggtgatatgtatggacgtaagattggtctgatgactgttgtcgtcatttactcaattgggatcatcatccaaatcgcctccattgacaaatggtatcaatatttcattggaagaatcatctcaggactgggcgttggtggtattacagttttggcgcctatgctaatttctgaagtgtcgcctaagcagttgcgtggtactctggtttcatgttaccaattaatgatcactttcggtatctttttgggatattgtactaattttggtaccaagaattactcaaactctgtccaatggagggtaccattaggcttatgctttgcatggtctatttttatgattgttggtatgacgttcgttcctgaatccccacgttatctggtagaagtgggaaaaattgaagaggccaagcggtccttagcaagagctaacaaaaccactgaagactctcctttagtaactttagaaatggagaactatcagtcttctattgaagctgagagattggcgggctctgcttcttggggggaattggttactggtaagccccagatgttcagacgtacactaatgggtatgatgattcaatctttacaacagctgacaggtgacaattacttcttttactatggtactacaattttccaggctgttggtttggaagattcatttgaaactgctattgttttgggtgttgttaattttgtttcgacttttttctcgctatataccgtcgatcgttttggtcgtcgtaattgtttgttatggggctgtgtaggtatgatttgttgctatgtcgtctatgcctctgttggtgttaccagattatggccaaacggtcaagatcaaccatcttcaaagggtgctggtaactgtatgattgttttcgcatgtttctacattttctgtttcgctaccacttgggcccccgttgcctatgtccttatctctgagtcgtatcccttaagagtacgtggtaaagcaatgtcgattgcaagtgcctgtaactggatttgggggttcttgatcagttttttcactccatttattacttcagcaatcaatttctattatggctatgtctttatgggttgtatggtgttcgcatacttttatgtgttcttctttgttccagagacaaagggcttaacattagaagaagtcaacgaaatgtatgaagaaaatgtgctaccttggaagtctaccaaatggatcccaccatctaggagaacaacagattatgacctagacgctactagaaatgatccgagaccattttataaaaggatgttcactaaagaaaaataa

SEQ ID NO: 28 - protein, 592 aa, Saccharomyces cerevisiae
MSELENAHQGPLEGSATVSTNSNSYNEKSGNSTAPGTAGYNDNLAQAKPVSSYISHEGPPKDELEELQKEVDKQLEKKSKSDLLFVSVCCLMVAFGGFVFGWDTGTISGFVRQTDFIRRFGSTRANGTTYLSDVRTGLMVSIFNIGCAIGGIVLSKLGDMYGRKIGLMTVVVIYSIGIIIQIASIDKWYQYFIGRIISGLGVGGITVLAPMLISEVSPKQLRGTLVSCYQLMITFGIFLGYCTNFGTKNYSNSVQWRVPLGLCFAWSIFMIVGMTFVPESPRYLVEVGKIEEAKRSLARANKTTEDSPLVTLEMENYQSSIEAERLAGSASWGELVTGKPQMFRRTLMGMMIQSLQQLTGDNYFFYYGTTIFQAVGLEDSFETAIVLGVVNFVSTFFSLYTVDRFGRRNCLLWGCVGMICCYVVYASVGVTRLWPNGQDQPSSKGAGNCMIVFACFYIFCFATTWAPVAYVLISESYPLRVRGKAMSIASACNWIWGFLISFFTPFITSAINFYYGYVFMGCMVFAYFYVFFFVPETKGLTLEEVNEMYEENVLPWKSTKWIPPSRRTTDYDLDATRNDPRPFYKRMFTKEK

SEQ ID NO: 29 - DNA, 1713 bp, Saccharomyces cerevisiae
atgtcacaagacgctgctattgcagagcaaactcctgtggagcatctctctgctgttgactcagcctcccactcggttttatctacaccatcaaacaaggctgaaagagatgaaataaaagcttatggtgaaggtgaagagcacgaacctgtcgttgaaattccaaagagaccagcttctgcctatgtcactgtctctattatgtgtatcatgatcgcctttggtggtttcgttttcggttgggatactggtaccatttctggtttcatcaatcaaaccgatttcatcagaagatttggtatgaagcataaagatggtactaattatttgtctaaggttagaactggtttgattgtctccattttcaacattggttgtgccattggtggtattattctttccaaattgggtgatatgtacggtcgtaaggtgggtttgattgtcgttgttgtcatctacatcatcggtattattattcaaattgcatctatcaacaaatggtaccaatatttcatcggtagaattatttccggtttgggtgttggtggtattgccgttttatctcctatgttgatttctgaagtatccccaaagcatttaaggggtactttagtctcttgctaccaattgatgattactgccggtattttcttgggttactgtaccaacttcggtactaagaactactccaactctgtgcaatggagagttccattaggtttgtgttttgcctgggctttgtttatgattggtggtatgacatttgttccagagtctccacgttatttggctgaagtcggtaagatcgaagaagccaaacgttctattgccgtttctaacaaggttgctgttgatgatccatctgttttggctgaagtcgaagctgtcttggctggtgtagaggcagagaaattagctggtaatgcatcctggggtgaattgtttagtagcaagacaaaggtccttcagcgtttgatcatgggtgctatgattcaatctctacaacaattgacaggtgataactatttcttctactatggtactactattttcaaggctgttggtttgagtgactctttcgaaacctctattgtcttgggtattgttaactttgcttccacctttgttggtatttacgttgttgagagatatggtcgtcgtacttgtttgctatggggtgctgcatccatgactgcttgtatggttgtctatgcttccgtgggtgtcaccagattatggccaaatggtcaagaccaaccatcttccaagggtgctggtaactgtatgattgtctttgcctgtttctatattttctgttttgctactacatgggctccaattccttatgtcgttgtttctgaaactttcccattgagagtcaagtctaaggctatgtctattgctacagctgctaattggttgtggggtttcttgattggtttcttcactccatttattactggtgctattaacttctactacggttacgttttcatgggctgtttggtcttcatgttcttctatgttttgttagttgttccagaaactaagggtttgactttggaagaagtcaacaccatgtgggaagaaggtgttctaccatggaagtctgcctcatgggttccaccatctagaagaggtgccaactacgacgctgaagaaatggctcacgatgataagccattgtacaagagaatgttcagcaccaaataa

SEQ ID NO: 30 - protein, 570 aa, Saccharomyces cerevisiae
MSQDAAIAEQTPVEHLSAVDSASHSVLSTPSNKAERDEIKAYGEGEEHEPVVEIPKRPASAYVTVSIMCIMIAFGGFVFGWDTGTISGFINQTDFIRRFGMKHKDGTNYLSKVRTGLIVSIFNIGCAIGGIILSKLGDMYGRKVGLIVVVVIYIIGIIIQIASINKWYQYFIGRIISGLGVGGIAVLSPMLISEVSPKHLRGTLVSCYQLMITAGIFLGYCTNFGTKNYSNSVQWRVPLGLCFAWALFMIGGMTFVPESPRYLAEVGKIEEAKRSIAVSNKVAVDDPSVLAEVEAVLAGVEAEKLAGNASWGELFSSKTKVLQRLIMGAMIQSLQQLTGDNYFFYYGTTIFKAVGLSDSFETSIVLGIVNFASTFVGIYVVERYGRRTCLLWGAASMTACMVVYASVGVTRLWPNGQDQPSSKGAGNCMIVFACFYIFCFATTWAPIPYVVVSETFPLRVKSKAMSIATAANWLWGFLIGFFTPFITGAINFYYGYVFMGCLVFMFFYVLLVVPETKGLTLEEVNTMWEEGVLPWKSASWVPPSRRGANYDAEEMAHDDKPLYKRMFSTK

SEQ ID NO: 31 - DNA, 1713 bp, Saccharomyces cerevisiae
atgtcacaagacgctgctattgcagagcaaactcctgtggagcatctctctgctgttgactcagcctcccactcggttttatctacaccatcaaacaaggctgaaagagatgaaataaaagcttatggtgaaggtgaagagcacgaacctgtcgttgaaattccaaagagaccagcttctgcctatgtcactgtctctattatgtgtatcatgatcgcctttggtggtttcgttttcggttgggatactggtaccatttctggtttcatcaatcaaaccgatttcatcagaagatttggtatgaagcataaagatggtactaattatttgtctaaggttagaactggtttgattgtctccattttcaacattggttgtgccattggtggtattattctttccaaattgggtgatatgtacggtcgtaaggtgggtttgattgtcgttgttgtcatctacatcatcggtattattattcaaattgcatctatcaacaaatggtaccaatatttcatcggtagaattatttccggtttgggtgttggtggtattgccgttttatctcctatgttgatttctgaagtatccccaaagcatttaaggggtactttagtctcttgctaccaattgatgattactgccggtattttcttgggttactgtaccaacttcggtactaagaactactccaactctgtgcaatggagagttccattaggtttgtgttttgcctgggctttgtttatgattggtggtatgacatttgttccagagtctccacgttatttggctgaagtcggtaagatcgaagaagccaaacgttctattgccgtttctaacaaggttgctgttgatgatccatctgttttggctgaagtcgaagctgtcttggctggtgtagaggcagagaaattagctggtaatgcatcctggggtgaattgtttagtagcaagacaaaggtccttcagcgtttgatcatgggtgctatgattcaatctctacaacaattgacaggtgataactatttcttctactatggtactactattttcaaggctgttggtttgagtgactctttcgaaacctctattgtcttgggtattgttaactttgcttccacctttgttggtatttacgttgttgagagatatggtcgtcgtacttgtttgctatggggtgctgcatccatgactgcttgtatggttgtctatgcttccgtgggtgtcaccagattatggccaaatggtcaagaccaaccatcttccaagggtgctggtaactgtatgattgtctttgcctgtttctatattttctgttttgctactacatgggctccaattccttatgtcgttgtttctgaaactttcccattgagagtcaagtctaaggctatgtctattgctacagctgctaattggttgtggggtttcttgattggtttcttcactccatttattactggtgctattaacttctactacggttacgttttcatgggctgtttggtcttcatgttcttctatgttttgttagttgttccagaaactaagggtttgactttggaagaagtcaacaccatgtgggaagaaggtgttctaccatggaagtctgcctcatgggttccaccatccagaagaggtgccaactacgacgctgaagaaatgactcacgatgacaagccattgtacaagagaatgttcagcaccaaataa

SEQ ID NO: 32 - protein, 570 aa, Saccharomyces cerevisiae
MSQDAAIAEQTPVEHLSAVDSASHSVLSTPSNKAERDEIKAYGEGEEHEPVVEIPKRPASAYVTVSIMCIMIAFGGFVFGWDTGTISGFINQTDFIRRFGMKHKDGTNYLSKVRTGLIVSIFNIGCAIGGIILSKLGDMYGRKVGLIVVVVIYIIGIIIQIASINKWYQYFIGRIISGLGVGGIAVLSPMLISEVSPKHLRGTLVSCYQLMITAGIFLGYCTNFGTKNYSNSVQWRVPLGLCFAWALFMIGGMTFVPESPRYLAEVGKIEEAKRSIAVSNKVAVDDPSVLAEVEAVLAGVEAEKLAGNASWGELFSSKTKVLQRLIMGAMIQSLQQLTGDNYFFYYGTTIFKAVGLSDSFETSIVLGIVNFASTFVGIYVVERYGRRTCLLWGAASMTACMVVYASVGVTRLWPNGQDQPSSKGAGNCMIVFACFYIFCFATTWAPIPYVVVSETFPLRVKSKAMSIATAANWLWGFLIGFFTPFITGAINFYYGYVFMGCLVFMFFYVLLVVPETKGLTLEEVNTMWEEGVLPWKSASWVPPSRRGANYDAEEMTHDDKPLYKRMFSTK

SEQ ID NO: 33 - DNA, 1458 bp, Saccharomyces cerevisiae
atggttcatttaggtccaaagaaaccacaggctagaaagggttccatggctgatgtgcccaaggaattgatggatgaaattcatcagttggaagatatgtttacagttgacagcgagaccttgagaaaggttgttaagcactttatcgacgaattgaataaaggtttgacaaagaagggaggtaacattccaatgattcccggttgggtcatggaattcccaacaggtaaagaatctggtaactatttggccattgatttgggtggtactaacttaagagtcgtgttggtcaagttgagcggtaaccatacctttgacaccactcaatccaagtataaactaccacatgacatgagaaccactaagcaccaagaggagttatggtcctttattgccgactctttgaaggactttatggtcgagcaagaattgctaaacaccaaggacaccttaccattaggtttcaccttctcgtacccagcttcccaaaacaagattaacgaaggtattttgcaaagatggaccaagggtttcgatattccaaatgtcgaaggccacgatgtcgtcccattgctacaaaacgaaatttccaagagagagttgcctattgaaattgtagcattgattaatgatactgttggtactttaattgcctcatactacactgacccagagactaagatgggtgtgattttcggtactggtgtcaacggtgctttctatgatgttgtttccgatatcgaaaagttggagggcaaattagcagacgatattccaagtaactctccaatggctatcaattgtgaatatggttccttcgataatgaacatttggtcttgccaagaaccaagtacgatgttgctgtcgacgaacaatctccaagacctggtcaacaagcttttgaaaagatgacctccggttactacttgggtgaattgttgcgtctagtgttacttgaattaaacgagaagggcttgatgttgaaggatcaagatctaagcaagttgaaacaaccatacatcatggatacctcctacccagcaagaatcgaggatgatccatttgaaaacttggaagatactgatgacatcttccaaaaggactttggtgtcaagaccactctgccagaacgtaagttgattagaagactttgtgaattgatcggtaccagagctgctagattagctgtttgtggtattgccgctatttgccaaaagagaggttacaagactggtcacattgccgctgacggttctgtctataacaaatacccaggtttcaaggaagccgccgctaagggtttgagagatatctatggatggactggtgacgcaagcaaagatccaattacgattgttccagctgaggatggttcaggtgcaggtgctgctgttattgctgcattgtccgaaaaaagaattgccgaaggtaagtctcttggtatcattggcgcttaa

SEQ ID NO: 34 - protein, 485 aa, Saccharomyces cerevisiae
MVHLGPKKPQARKGSMADVPKELMDEIHQLEDMFTVDSETLRKVVKHFIDELNKGLTKKGGNIPMIPGWVMEFPTGKESGNYLAIDLGGTNLRVVLVKLSGNHTFDTTQSKYKLPHDMRTTKHQEELWSFIADSLKDFMVEQELLNTKDTLPLGFTFSYPASQNKINEGILQRWTKGFDIPNVEGHDVVPLLQNEISKRELPIEIVALINDTVGTLIASYYTDPETKMGVIFGTGVNGAFYDVVSDIEKLEGKLADDIPSNSPMAINCEYGSFDNEHLVLPRTKYDVAVDEQSPRPGQQAFEKMTSGYYLGELLRLVLLELNEKGLMLKDQDLSKLKQPYIMDTSYPARIEDDPFENLEDTDDIFQKDFGVKTTLPERKLIRRLCELIGTRAARLAVCGIAAICQKRGYKTGHIAADGSVYNKYPGFKEAAAKGLRDIYGWTGDASKDPITIVPAEDGSGAGAAVIAALSEKRIAEGKSLGIIGA

SEQ ID NO: 35 - DNA, 1461 bp, Saccharomyces cerevisiae
atggttcatttaggtccaaaaaaaccacaagccagaaagggttccatggccgatgtgccaaaggaattgatgcaacaaattgagaattttgaaaaaattttcactgttccaactgaaactttacaagccgttaccaagcacttcatttccgaattggaaaagggtttgtccaagaagggtggtaacattccaatgattccaggttgggttatggatttcccaactggtaaggaatccggtgatttcttggccattgatttgggtggtaccaacttgagagttgtcttagtcaagttgggcggtgaccgtacctttgacaccactcaatctaagtacagattaccagatgctatgagaactactcaaaatccagacgaattgtgggaatttattgccgactctttgaaagcttttattgatgagcaattcccacaaggtatctctgagccaattccattgggtttcaccttttctttcccagcttctcaaaacaaaatcaatgaaggtatcttgcaaagatggactaaaggttttgatattccaaacattgaaaaccacgatgttgttccaatgttgcaaaagcaaatcactaagaggaatatcccaattgaagttgttgctttgataaacgacactaccggtactttggttgcttcttactacactgacccagaaactaagatgggtgttatcttcggtactggtgtcaatggtgcttactacgatgtttgttccgatatcgaaaagctacaaggaaaactatctgatgacattccaccatctgctccaatggccatcaactgtgaatacggttccttcgataatgaacatgtcgttttgccaagaactaaatacgatatcaccattgatgaagaatctccaagaccaggccaacaaacctttgaaaaaatgtcttctggttactacttaggtgaaattttgcgtttggccttgatggacatgtacaaacaaggtttcatcttcaagaaccaagacttgtctaagttcgacaagcctttcgtcatggacacttcttacccagccagaatcgaggaagatccattcgagaacctagaagataccgatgacttgttccaaaatgagttcggtatcaacactactgttcaagaacgtaaattgatcagacgtttatctgaattgattggtgctagagctgctagattgtccgtttgtggtattgctgctatctgtcaaaagagaggttacaagaccggtcacatcgctgcagacggttccgtttacaacagatacccaggtttcaaagaaaaggctgccaatgctttgaaggacatttacggctggactcaaacctcactagacgactacccaatcaagattgttcctgctgaagatggttccggtgctggtgccgctgttattgctgctttggcccaaaaaagaattgctgaaggtaagtccgttggtatcatcggtgcttaa

SEQ ID NO: 36 - protein, 486 aa, Saccharomyces cerevisiae
MVHLGPKKPQARKGSMADVPKELMQQIENFEKIFTVPTETLQAVTKHFISELEKGLSKKGGNIPMIPGWVMDFPTGKESGDFLAIDLGGTNLRVVLVKLGGDRTFDTTQSKYRLPDAMRTTQNPDELWEFIADSLKAFIDEQFPQGISEPIPLGFTFSFPASQNKINEGILQRWTKGFDIPNIENHDVVPMLQKQITKRNIPIEVVALINDTTGTLVASYYTDPETKMGVIFGTGVNGAYYDVCSDIEKLQGKLSDDIPPSAPMAINCEYGSFDNEHVVLPRTKYDITIDEESPRPGQQTFEKMSSGYYLGEILRLALMDMYKQGFIFKNQDLSKFDKPFVMDTSYPARIEEDPFENLEDTDDLFQNEFGINTTVQERKLIRRLSELIGARAARLSVCGIAAICQKRGYKTGHIAADGSVYNRYPGFKEKAANALKDIYGWTQTSLDDYPIKIVPAEDGSGAGAAVIAALAQKRIAEGKSVGIIGA

SEQ ID NO: 37 - DNA, 1503 bp, Saccharomyces cerevisiae
atgtcattcgacgacttacacaaagccactgagagagcggtcatccaggccgtggaccagatctgcgacgatttcgaggttacccccgagaagctggacgaattaactgcttacttcatcgaacaaatggaaaaaggtctagctccaccaaaggaaggccacacattggcctcggacaaaggtcttcctatgattccggcgttcgtcaccgggtcacccaacgggacggagcgcggtgttttactagccgccgacctgggtggtaccaatttccgtatatgttctgttaacttgcatggagatcatactttctccatggagcaaatgaagtccaagattcccgatgatttgctagacgatgagaacgtcacatctgacgacctgtttgggtttctagcacgtcgtacactggcctttatgaagaagtatcacccggacgagttggccaagggtaaagacgccaagcccatgaaactggggttcactttctcataccctgtagaccagacctctctaaactccgggacattgatccgttggaccaagggtttccgcatcgcggacaccgtcggaaaggatgtcgtgcaattgtaccaggagcaattaagcgctcagggtatgcctatgatcaaggttgttgcattaaccaacgacaccgtcggaacgtacctatcgcattgctacacgtccgataacacggactcaatgacgtccggagaaatctcggagccggtcatcggatgtattttcggtaccggtaccaatgggtgctatatggaggagatcaacaagatcacgaagttgccacaggagttgcgtgacaagttgataaaggagggtaagacacacatgatcatcaatgtcgaatgggggtccttcgataatgagctcaagcacttgcctactactaagtatgacgtcgtaattgaccagaaactgtcaacgaacccgggatttcacttgtttgaaaaacgtgtctcagggatgttcttgggtgaggtgttgcgtaacattttagtggacttgcactcgcaaggcttgcttttgcaacagtacaggtccaaggaacaacttcctcgccacttgactacacctttccagttgtcatccgaagtgctgtcgcatattgaaattgacgactcgacaggtctacgtgaaacagagttgtcattattacagagtctcagactgcccaccactccaacagagcgtgttcaaattcaaaaattggtgcgcgcgatttctaggagatctgcgtatttagccgccgtgccgcttgccgcgatattgatcaagacaaatgctttgaacaagagatatcatggtgaagtcgagatcggttgtgatggttccgttgtggaatactaccccggtttcagatctatgctgagacacgccttagccttgtcacccttgggtgccgagggtgagaggaaggtgcacttgaagattgccaaggatggttccggagtgggtgccgccttgtgtgcgcttgtagcatga

SEQ ID NO: 38 - protein, 500 aa, Saccharomyces cerevisiae
MSFDDLHKATERAVIQAVDQICDDFEVTPEKLDELTAYFIEQMEKGLAPPKEGHTLASDKGLPMIPAFVTGSPNGTERGVLLAADLGGTNFRICSVNLHGDHTFSMEQMKSKIPDDLLDDENVTSDDLFGFLARRTLAFMKKYHPDELAKGKDAKPMKLGFTFSYPVDQTSLNSGTLIRWTKGFRIADTVGKDVVQLYQEQLSAQGMPMIKVVALTNDTVGTYLSHCYTSDNTDSMTSGEISEPVIGCIFGTGTNGCYMEEINKITKLPQELRDKLIKEGKTHMIINVEWGSFDNELKHLPTTKYDVVIDQKLSTNPGFHLFEKRVSGMFLGEVLRNILVDLHSQGLLLQQYRSKEQLPRHLTTPFQLSSEVLSHIEIDDSTGLRETELSLLQSLRLPTTPTERVQIQKLVRAISRRSAYLAAVPLAAILIKTNALNKRYHGEVEIGCDGSVVEYYPGFRSMLRHALALSPLGAEGERKVHLKIAKDGSGVGAALCALVA

(19) United States
(12) Patent Application Publication
(10) Pub. No.: US 2021/0269832 A1
(43) Pub. Date: Sep. 2, 2021

(54) METHODS AND COMPOSITIONS FOR ENHANCED ETHANOL PRODUCTION

(71) Applicant: DANISCO US INC, Palo Alto, CA (US)

(72) Inventors: ANDREI MIASNIKOV, UNION CITY, CA (US); GEETHA VEERAMUTHU, SUNNYVALE, CA (US)

(21) Appl. No.: 17/173,412

(22) Filed: Feb. 11, 2021

Related U.S. Application Data
(60) Provisional application No. 62/982,290, filed on Feb. 27, 2020.

(51) Int. Cl. C12P 7/06; C12N 15/10

(52) U.S. Cl. CPC C12P 7/06; C12R 2001/85; C12N 15/102

(57) ABSTRACT

The present disclosure is related to the fields of biology, molecular biology, genetics, microbial fermentation, alcohol production and the like. The present compositions and methods relate to yeast strains comprising genetic modifications that results in modified yeast strains thereof comprising enhanced stress tolerance. Certain embodiments of the disclosure are therefore related to compositions and methods for increasing the efficiency of alcohol production using such modified yeast strains in fermentation reactions/processes.

### TECHNICAL FIELD

The present disclosure is generally related to the fields of biology, molecular biology, genetics, microbial fermentation, alcohol production and the like. More particularly, the present compositions and methods relate to yeast strains (cells) comprising genetic modifications that results in modified yeast strains thereof comprising enhanced stress tolerance. Certain embodiments of the disclosure are therefore related to compositions and methods for increasing the efficiency of alcohol production using such modified yeast strains in fermentation reactions/processes. Such modified yeast strains of the disclosure are well-suited for use in alcohol production to reduce fermentation time, increase yields and the like.

### CROSS REFERENCE TO RELATED APPLICATIONS

This application claims benefit to U.S. Provisional Application No. 62/982,290, filed Feb. 27, 2020, which is hereby incorporated by reference in its entirety.

### REFERENCE TO A SEQUENCE LISTING

The contents of the electronic submission of the text file Sequence Listing, named “NB41718-US_SequenceListing.txt” was created on Jan. 27, 2021 and is 84 KB in size, which is hereby incorporated by reference in its entirety.

### BACKGROUND OF INVENTION

Many countries make fuel alcohol from fermentable substrates, such as corn starch, sugar cane, cassava, molasses and the like. According to the Renewable Fuel Association (Washington D.C., United States), 2015 fuel ethanol production was close to 15 billion gallons in the United States, alone. In view of the large amount of alcohol produced in the world, even a minor increase in the efficiency of a fermenting microorganism can result in a tremendous increase in the amount of available alcohol. Accordingly, the need exists for microorganisms that are more efficient at producing alcohol.

### SUMMARY OF THE DISCLOSURE

The present disclosure is generally related to the methods and compositions for the biological production of ethanol. More particularly, certain embodiments are related to genetically modified yeast strains (cells) comprising enhanced ethanol production phenotypes. Thus, certain embodiments of the disclosure are related to a modified yeast cell derived from a parental yeast cell, wherein the modified cell comprises an attenuated ability to transport glucose and/or an attenuated ability to catalyze the phosphorylation of glucose into glucose 6-phosphate compared to the parental cell, wherein the modified cell comprises an enhanced stress tolerance phenotype compared to the parental cell when fermented under identical conditions for the production of ethanol. In certain embodiments, the attenuated ability to transport glucose comprises at least one genetic alteration that causes the modified cell to produce a decreased amount of a functional HXT1 polypeptide, a decreased amount of a functional HXT2 polypeptide, a decreased amount of a functional HXT3 polypeptide, a decreased amount of a functional HXT4 polypeptide, a decreased amount of a functional HXT5 polypeptide, a decreased amount of a functional HXT6 polypeptide and/or a decreased amount of a functional HXT7 polypeptide compared to the parental cell. In certain embodiments, the genetic alteration comprises a disruption of the HXT1 gene, the HXT2 gene, the HXT3 gene, the HXT4 gene, the HXT5 gene, the HXT6 gene and/or the HXT7 gene present in the parental cell. In certain embodiments, disruption of the HXT1 gene, the HXT2 gene, the HXT3 gene, the HXT4 gene, the HXT5 gene, the HXT6 gene and/or the HXT7 gene is the result of deletion of all or part of the HXT1 gene, the HXT2 gene, the HXT3 gene, the HXT4 gene, the HXT5 gene, the HXT6 gene and/or the HXT7 gene. In other embodiments, disruption of the HXT1 gene, the HXT2 gene, the HXT3 gene, the HXT4 gene, the HXT5 gene, the HXT6 gene and/or the HXT7 gene is the result of deletion of a portion of genomic DNA comprising the HXT1 gene, the HXT2 gene, the HXT3 gene, the HXT4 gene, the HXT5 gene, the HXT6 gene and/or the HXT7 gene. In other embodiments, disruption of the HXT1 gene, the HXT2 gene, the HXT3 gene, the HXT4 gene, the HXT5 gene, the HXT6 gene and/or the HXT7 gene is the result of mutagenesis of the HXT1 gene, the HXT2 gene, the HXT3 gene, the HXT4 gene, the HXT5 gene, the HXT6 gene and/or the HXT7 gene. In other embodiments, the attenuated ability to catalyze the phosphorylation of glucose into glucose 6-phosphate comprises a genetic alteration that causes the modified cell to produce a decreased amount of a functional HXK1 polypeptide, a decreased amount of a functional HXK2 polypeptide, and/or a decreased amount of a functional GLK1 polypeptide compared to the parental cell. In certain embodiments, the genetic alteration comprises a disruption of the HXK1 gene, the HXK2 gene, and/or the GLK1 gene present in the parental cell. In certain embodiments, the genetic alteration comprises a disruption of the HXK1 gene, the HXK2 gene, and/or the GLK1 gene present in the parental cell. In other embodiments, disruption of the HXK1 gene, the HXK2 gene and/or the GLK1 gene is the result of deletion of all or part of the HXK1 gene, the HXK2 gene and/or the GLK1. In certain other embodiments, disruption of the HXK1 gene, the HXK2 gene and/or the GLK1 gene is the result of deletion of a portion of genomic DNA comprising the HXK1 gene, the HXK2 gene and/or the GLK1 gene. In other embodiments, disruption of the HXK1 gene, the HXK2 gene and/or the GLK1 gene is the result of mutagenesis of the HXK1 gene, the HXK2 gene and/or the GLK1 gene. In certain embodiments, the modified cell does not produce a functional HXT1 polypeptide, a functional HXT2 polypeptide, a functional HXT3 polypeptide, a functional HXT4 polypeptide, a functional HXT5 polypeptide, a functional HXT6 polypeptide and/or a functional HXT7 polypeptide. In other embodiments, the modified cell does not produce a functional HXK1 polypeptide, a functional HXK2 polypeptide, and/or a functional GLK1 polypeptide. In certain embodiments, the cell further comprises an exogenous gene encoding a carbohydrate processing enzyme. In particular embodiments, the yeast cell is a Saccharomyces spp. In another embodiment, the enhanced stress tolerance phenotype is an enhanced ability to ferment glucose to ethanol at an elevated fermentation temperature. In certain embodiments, the elevated temperature is 32° C. In certain other embodiments, the elevated temperature is 33° C. In certain other embodiments, the elevated temperature is 34° C. In certain other embodiments, the elevated temperature is 35° C. or higher. In certain embodiments, the enhanced stress tolerance phenotype is an enhanced ability to finish fermentation of glucose to ethanol in the presence of a high dry solids (DS) liquefact concentration. In another embodiment, the dry solids (DS) liquefact concentration is about 32% DS. In certain other embodiments, the DS liquefact concentration is about 33% DS. In certain other embodiments, the DS liquefact concentration is about 34% DS or higher. In other embodiments, the enhanced stress tolerance phenotype is an enhanced rate of ethanol production. In certain other embodiments, the enhanced stress tolerance phenotype is an increased ethanol yield.

In other embodiments, the disclosure is directed to a genetically modified yeast cell derived from a parental yeast cell, wherein the modified cell comprises an attenuated ability to transport glucose and/or an attenuated ability to catalyze the phosphorylation of glucose into glucose 6-phosphate compared to the parental cell, wherein the modified cell produces during fermentation an increased amount of ethanol compared to parental cell when fermented under identical conditions for the production of ethanol. Thus, in certain embodiments, the attenuated ability to transport glucose comprises at least one genetic alteration that causes the modified cell to produce a decreased amount of a functional HXT1 polypeptide, a decreased amount of a functional HXT2 polypeptide, a decreased amount of a functional HXT3 polypeptide, a decreased amount of a functional HXT4 polypeptide, a decreased amount of a functional HXT5 polypeptide, a decreased amount of a functional HXT6 polypeptide and/or a decreased amount of a functional HXT7 polypeptide compared to the parental cell. In certain embodiments, the genetic alteration comprises a disruption of the HXT1 gene, the HXT2 gene, the HXT3 gene, the HXT4 gene, the HXT5 gene, the HXT6 gene and/or the HXT7 gene present in the parental cell. In another embodiment, disruption of the HXT1 gene, the HXT2 gene, the HXT3 gene, the HXT4 gene, the HXT5 gene, the HXT6 gene and/or the HXT7 gene is the result of deletion of all or part of the HXT1 gene, the HXT2 gene, the HXT3 gene, the HXT4 gene, the HXT5 gene, the HXT6 gene and/or the HXT7 gene. In certain other embodiments, disruption of the HXT1 gene, the HXT2 gene, the HXT3 gene, the HXT4 gene, the HXT5 gene, the HXT6 gene and/or the HXT7 gene is the result of deletion of a portion of genomic DNA comprising the HXT1 gene, the HXT2 gene, the HXT3 gene, the HXT4 gene, the HXT5 gene, the HXT6 gene and/or the HXT7 gene. In another embodiment, disruption of the HXT1 gene, the HXT2 gene, the HXT3 gene, the HXT4 gene, the HXT5 gene, the HXT6 gene and/or the HXT7 gene is the result of mutagenesis of the HXT1 gene, the HXT2 gene, the HXT3 gene, the HXT4 gene, the HXT5 gene, the HXT6 gene and/or the HXT7 gene. In certain other embodiments, the modified cell does not produce a functional HXT1 polypeptide, a functional HXT2 polypeptide, a functional HXT3 polypeptide, a functional HXT4 polypeptide, a functional HXT5 polypeptide, a functional HXT6 polypeptide and/or a functional HXT7 polypeptide. In certain other embodiments, the attenuated ability to catalyze the phosphorylation of glucose into glucose 6-phosphate comprises a genetic alteration that causes the modified cell to produce a decreased amount of a functional HXK1 polypeptide, a decreased amount of a functional HXK2 polypeptide, and/or a decreased amount of a functional GLK1 polypeptide compared to the parental cell. Thus, in certain other embodiments, the modified cell does not produce a functional HXK1 polypeptide, a functional HXK2 polypeptide, and/or a functional GLK1 polypeptide. In other embodiments, the attenuated ability to catalyze the phosphorylation of glucose into glucose 6-phosphate comprises a genetic alteration that causes the modified cell to produce a decreased amount of a functional HXK1 polypeptide, a decreased amount of a functional HXK2 polypeptide, and/or a decreased amount of a functional GLK1 polypeptide compared to the parental cell. In certain embodiments, the genetic alteration comprises a disruption of the HXK1 gene, the HXK2 gene, and/or the GLK1 gene present in the parental cell. In other embodiments, disruption of the HXK1 gene, the HXK2 gene and/or the GLK1 gene is the result of deletion of all or part of the HXK1 gene, the HXK2 gene and/or the GLK1. In other embodiments, disruption of the HXK1 gene, the HXK2 gene and/or the GLK1 gene is the result of deletion of a portion of genomic DNA comprising the HXK1 gene, the HXK2 gene and/or the GLK1 gene. In certain embodiments, disruption of the HXK1 gene, the HXK2 gene and/or the GLK1 gene is the result of mutagenesis of the HXK1 gene, the HXK2 gene and/or the GLK1 gene. In certain embodiments, the modified cell does not produce a functional HXK1 polypeptide, a functional HXK2 polypeptide, and/or a functional GLK1 polypeptide. In certain other embodiments, the cell further comprises an exogenous gene encoding a carbohydrate processing enzyme. In certain embodiments, the yeast cell is a Saccharomyces spp. Thus, in certain other embodiments, the modified cell produces the increased amount of ethanol at an increased rate, relative to the parental cell when fermented under identical conditions for the production of ethanol. In certain embodiments, the increased rate of ethanol production is completed in about 55 hours, relative to the parental when fermented under identical conditions. In other embodiments, the increased rate of ethanol production is completed in about 56 to 70 hours, relative to the parental when fermented under identical conditions. In related embodiments, the modified cell further comprises an enhanced ability to ferment glucose to ethanol at elevated temperatures. In particular embodiments, the modified cell further comprises an enhanced ability to ferment a high dry solids (DS) liquefact into ethanol.

In certain other embodiments, the disclosure is related to a method for producing a modified yeast cell comprising introducing one or more genetic alterations into a parental yeast cell, which genetic alteration reduces or prevents the production of a functional HXT1 polypeptide, a functional HXT2 polypeptide, a functional HXT3 polypeptide, a functional HXT4 polypeptide, a functional HXT5 polypeptide, a functional HXT6 polypeptide, a functional HXT7 polypeptide, a functional HXK1 polypeptide, a functional HXK2 polypeptide and/or a functional GLK1 polypeptide compared to the parental cell, thereby producing a modified cell that produces during fermentation an increased amount of ethanol compared to the parental cells under equivalent fermentation conditions. In certain embodiments, the genetic alteration comprises disrupting the HXT1 gene, the HXT2 gene, the HXT3 gene, the HXT4 gene, the HXT5 gene, the HXT6 gene, the HXT7 gene, the HXK1 gene, the HXK2 gene and/or the GLK1 gene in the parental cells by genetic manipulation. In other embodiments, the genetic alteration comprises deleting the HXT1 gene, the HXT2 gene, the HXT3 gene, the HXT4 gene, the HXT5 gene, the HXT6 gene, the HXT7 gene, the HXK1 gene, the HXK2 gene and/or the GLK1 gene in the parental cells using genetic manipulation. In yet other embodiments, the genetic alteration comprises down-regulating the HXT1 gene, the HXT2 gene, the HXT3 gene, the HXT4 gene, the HXT5 gene, the HXT6 gene, the HXT7 gene, the HXK1 gene, the HXK2 gene and/or the GLK1 gene in the parental cells using genetic manipulation. In certain embodiments, down-regulating a gene comprises replacing the native gene promoter with a reduced activity promoter, or truncating or deleting the native gene promoter sequence, or truncating or deleting the native gene 5′-UTR sequence, or truncating or deleting the native gene 3′-UTR sequence in the parental cells using genetic manipulation. In certain embodiments, the modified yeast cell is a Saccharomyces spp. In certain other embodiments, the amount of ethanol produced by the modified yeast cell and the parental yeast cell is measured at 55 hours following inoculation of a hydrolyzed starch substrate comprising 32%-36% dissolved solids (DS). In other embodiments, the increased amount of ethanol produced by the modified cell is increased at a fermentation temperature of 32° C. to 35° C. In other embodiments, the increased rate of ethanol produced (by the modified yeast cell) is an increased rate relative to the parental cell rate of ethanol produced. In certain other embodiments, the increased amount of ethanol produced by the modified yeast cell is in the presence of about a 32%-36% dissolved solids.

Thus, certain other embodiments of the disclosure are related to modified yeast strains (cells) produced by any of the methods and/or compositions described herein. Certain other embodiments are therefore related to methods for producing increased amounts of ethanol in a yeast fermentation process, such methods comprising fermenting a modified yeast cell of the disclosure under suitable conditions for the production of ethanol, wherein the modified cell produces an increased amount of ethanol relative to the parental cell when fermented under identical conditions.

### BRIEF DESCRIPTION OF THE BIOLOGICAL SEQUENCES

SEQ ID NO: 1 is the polynucleotide sequence of a HXT1 disruption cassette.

SEQ ID NO: 2 is the polynucleotide sequence of a HXT3 disruption cassette.

SEQ ID NO: 3 is the polynucleotide sequence of a HXT4 disruption cassette.

SEQ ID NO: 4 is the polynucleotide sequence of a HXK2 disruption cassette.

SEQ ID NO: 5 is a HXT1_MAP_D1 nucleic acid primer sequence.

SEQ ID NO: 6 is a HXT1_MAP_R1 nucleic acid primer sequence.

SEQ ID NO: 7 is a HXT1_MAP_R2 nucleic acid primer sequence.

SEQ ID NO: 8 is a HXT3_MAP_D1 nucleic acid primer sequence.

SEQ ID NO: 9 is a HXT3_MAP_R1 nucleic acid primer sequence.

SEQ ID NO: 10 is a HXT3_MAP_R2 nucleic acid primer sequence.

SEQ ID NO: 11 is a HXT4_MAP_D1 nucleic acid primer sequence.

SEQ ID NO: 12 is a HXT4_MAP_R1 nucleic acid primer sequence.

SEQ ID NO: 13 is a HXT4_MAP_R2 nucleic acid primer sequence.

SEQ ID NO: 14 is a HXK2_MAP_D1 nucleic acid primer sequence.

SEQ ID NO: 15 is a HXK2_MAP_R1 nucleic acid primer sequence.

SEQ ID NO: 16 is a HXK2_MAP_R2 nucleic acid primer sequence.

SEQ ID NO: 17 is a URA3_MAP_D1 nucleic acid primer sequence.

SEQ ID NO: 18 is a URA3_MAP_R1 nucleic acid primer sequence.

SEQ ID NO: 19 is the nucleic acid sequence of a S. cerevisiae HXT1 gene.

SEQ ID NO: 20 is the amino acid sequence of the HXT1 protein encoded by S. cerevisiae HXT1 gene.

SEQ ID NO: 21 is the nucleic acid sequence of a S. cerevisiae HXT2 gene.

SEQ ID NO: 22 is the amino acid sequence of the HXT2 protein encoded by S. cerevisiae HXT2 gene.

SEQ ID NO: 23 is the nucleic acid sequence of a S. cerevisiae HXT3 gene.

SEQ ID NO: 24 is the amino acid sequence of the HXT3 protein encoded by S. cerevisiae HXT3 gene.

SEQ ID NO: 25 is the nucleic acid sequence of a S. cerevisiae HXT4 gene.

SEQ ID NO: 26 is the amino acid sequence of the HXT4 protein encoded by S. cerevisiae HXT4 gene.

SEQ ID NO: 27 is the nucleic acid sequence of a S. cerevisiae HXT5 gene.

SEQ ID NO: 28 is the amino acid sequence of the HXT5 protein encoded by S. cerevisiae HXT5 gene.

SEQ ID NO: 29 is the nucleic acid sequence of a S. cerevisiae HXT6 gene.

SEQ ID NO: 30 is the amino acid sequence of the HXT6 protein encoded by S. cerevisiae HXT6 gene.

SEQ ID NO: 31 is the nucleic acid sequence of a S. cerevisiae HXT7 gene.

SEQ ID NO: 32 is the amino acid sequence of the HXT7 protein encoded by S. cerevisiae HXT7 gene.

SEQ ID NO: 33 is the nucleic acid sequence of a S. cerevisiae HXK1 gene.

SEQ ID NO: 34 is the amino acid sequence of the HXK1 protein encoded by S. cerevisiae HXK1 gene.

SEQ ID NO: 35 is the nucleic acid sequence of a S. cerevisiae HXK2 gene.

SEQ ID NO: 36 is the amino acid sequence of the HXK2 protein encoded by S. cerevisiae HXK2 gene.

SEQ ID NO: 37 is the nucleic acid sequence of a S. cerevisiae GLK1 gene.

SEQ ID NO: 38 is the amino acid sequence of the GLK1 protein encoded by S. cerevisiae GLK1 gene.

### BRIEF DESCRIPTION OF THE DRAWINGS

FIG. 1A-1D show the design and various stages of the gene disruption process using a double crossover disruption cassette. FIG. 1A shows the chromosomal region around the wild-type HXT1 gene in FermaxGold strain, with regions used for disruption cassette design highlighted; FIG. 1B shows the synthetic (HXT1) disruption cassette; FIG. 1C shows the chromosomal region of FermaxGold with the HXT1 gene disrupted by URA3 and FIG. 1D shows the same chromosomal region after excision of URA3 marker gene.

FIG. 2 presents the end-of-run (66 hours) glucose and ethanol concentrations in high temperature (35° C.) SSFs with wild-type yeast strain FermaxGold and the modified strains carrying various single allele and double allele deletions in glucose uptake and phosphorylation genes (see, TABLE 3).

FIG. 3 shows the fermentation rate, measured as weight loss due to CO₂ emission. More particularly, the data for the wild-type yeast strain FermaxGold and the modified strains carrying various single allele and double allele deletions in glucose uptake and phosphorylation genes (TABLE 3) are presented in FIG. 3. The data on the three graphs are from a single experiment, spread over three panels as indicated for better visibility, wherein the control strain (FermaxGold) weight loss curve is repeated on each panel.

FIG. 4 shows the end-of-run (66 hours) glucose and ethanol concentrations in high dry solids (36% DS) SSFs with the wild-type yeast strain (FermaxGold) and the modified strains carrying various single allele and double allele deletions in glucose uptake and phosphorylation genes (TABLE 3).

### DETAILED DESCRIPTION

The present compositions and methods relate to modified yeast strains (cells) demonstrating increased ethanol production efficiency compared to their parental cells. As described herein, when used for ethanol production, the modified cells allow for increased ethanol yields and increased rates of ethanol production (e.g., shorter fermentation times) and the like, thereby increasing the supply of ethanol for world consumption.

### I. Definitions

Prior to describing the present compositions and methods in detail, the following terms are defined for clarity. Terms not defined should be accorded their ordinary meanings as used in the relevant art. Unless defined otherwise, all technical and scientific terms used herein have the same meaning as commonly understood by one of ordinary skill in the art to which the present compositions and methods apply.

All publications and patents cited in this specification are herein incorporated by reference.

Where a range of values is provided, it is understood that each intervening value, to the tenth of the unit of the lower limit unless the context clearly dictates otherwise, between the upper and lower limit of that range and any other stated or intervening value in that stated range, is encompassed within the present compositions and methods. The upper and lower limits of these smaller ranges may independently be included in the smaller ranges and are also encompassed within the present compositions and methods, subject to any specifically excluded limit in the stated range. Where the stated range includes one or both of the limits, ranges excluding either or both of those included limits are also included in the present compositions and methods.

Certain ranges are presented herein with numerical values being preceded by the term “about”. The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number. For example, in connection with a numerical value, the term “about” refers to a range of −10% to +10% of the numerical value, unless the term is otherwise specifically defined in context. In another example, the phrase a “pH value of about 6” refers to pH values of from 5.4 to 6.6, unless the pH value is specifically defined otherwise.

The headings provided herein are not limitations of the various aspects or embodiments of the present compositions and methods which can be had by reference to the specification as a whole. Accordingly, the terms defined immediately below are more fully defined by reference to the specification as a whole.

In accordance with this Detailed Description, the following abbreviations and definitions apply. Note that the singular forms “a,” “an,” and “the” include plural referents unless the context clearly dictates otherwise. Thus, for example, reference to “an enzyme” includes a plurality of such enzymes, and reference to “the dosage” includes reference to one or more dosages and equivalents thereof known to those skilled in the art, and so forth.

It is further noted that the claims may be drafted to exclude any optional element. As such, this statement is intended to serve as antecedent basis for use of such exclusive terminology as “solely”, “only”, “excluding”, “not including” and the like in connection with the recitation of claim elements, or use of a “negative” limitation.

It is further noted that the term “comprising”, as used herein, means “including, but not limited to”, the component(s) after the term “comprising”. The component(s) after the term “comprising” are required or mandatory, but the composition comprising the component(s) may further include other non-mandatory or optional component(s).

It is also noted that the term “consisting of,” as used herein, means “including and limited to”, the component(s) after the term “consisting of”. The component(s) after the term “consisting of” are therefore required or mandatory, and no other component(s) are present in the composition.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

As used herein, an industrial yeast strain named “FermaxGold” is a diploid yeast strain comprising a deletion in URA3 gene rending the strain a uridine auxotroph.

As used herein, a yeast gene named “HXT1” (SEQ ID NO: 19) encodes a “hexose transporter 1 protein” (HXT1; SEQ ID NO: 20), a yeast gene named “HXT2” (SEQ ID NO: 21) encodes a “hexose transporter 2 protein” (HXT2; SEQ ID NO: 22), a yeast gene named “HXT3” (SEQ ID NO: 23) encodes a “hexose transporter 3 protein” (HXT3; SEQ ID NO: 24), a yeast gene named “HXT4” (SEQ ID NO: 25) encodes a “hexose transporter 4 protein” (HXT4; SEQ ID NO: 26), a yeast gene named “HXT5” (SEQ ID NO: 27) encodes a “hexose transporter 5 protein” (HXT5; SEQ ID NO: 28), a yeast gene named “HXT6” (SEQ ID NO: 29) encodes a “hexose transporter 6 protein” (HXT6; SEQ ID NO: 30) and a yeast gene named “HXT7” (SEQ ID NO: 31) encodes a “hexose transporter 7 protein” (HXT7; SEQ ID NO: 32).

In certain embodiments, a yeast HXT1 gene comprises about 85% to 99% sequence identity to SEQ ID NO: 19 and encodes a functional HXT1 protein; a yeast HXT2 gene comprises about 85% to 99% sequence identity to SEQ ID NO: 21 and encodes a functional HXT2 protein; a yeast HXT3 gene comprises about 85% to 99% sequence identity to SEQ ID NO: 23 and encodes a functional HXT3 protein; a yeast HXT4 gene comprises about 85% to 99% sequence identity to SEQ ID NO: 25 and encodes a functional HXT4 protein; a yeast HXT5 gene comprises about 85% to 99% sequence identity to SEQ ID NO: 27 and encodes a functional HXT5 protein; a yeast HXT6 gene comprises about 85% to 99% sequence identity to SEQ ID NO: 27 and encodes a functional HXT6 protein; and a yeast HXT7 gene comprises about 85% to 99% sequence identity to SEQ ID NO: 31 and encodes a functional HXT7 protein.

As used herein, a “functional” hexose transporter 1 protein (HXT1), a “functional” hexose transporter 2 protein (HXT2), a “functional” hexose transporter 3 protein (HXT3), a “functional” hexose transporter 4 protein (HXT4), a “functional” hexose transporter 5 protein (HXT5), a “functional” hexose transporter 6 protein” (HXT6) and a “functional” hexose transporter 7 protein (HXT7) comprise substrate specific (i.e., hexose sugars, e.g., glucose, fructose) transmembrane transporter activity. Thus, as used herein, a functional HXT1 protein, a functional HXT2 protein, a functional HXT3 protein, a functional HXT4 protein, a functional HXT5 protein, a functional HXT6 protein and/or a functional HXT7 protein refer to hexose transporter proteins capable of transporting exogenous hexose sugars (e.g., glucose) into the cytoplasm of a yeast cell.

As used herein, a hexose transporter (i.e., HXT1-HXT7) protein comprising “reduced (decreased) hexose transporter activity” or “reduced (decreased) hexose transporter function” may be used interchangeably, and refer to a hexose transporter (i.e., HXT1-HXT7) protein with a reduced (decreased) ability to transport exogenous hexose sugars (e.g., glucose) into the cytoplasm of a yeast cell relative to a functional hexose transporter (i.e., HXT1-HXT7) protein.

As used herein, yeast cells comprising an “attenuated ability to transport glucose” refers to a yeast cell that has been engineered (constructed) to decrease the glucose uptake (transport) rate.

The engineering can be done by a variety of methods known to those skilled in the art of microbial strain engineering/construction. These methods include, but are not limited to, inactivation of the genes encoding the hexose transporters (e.g., HXT1-HXT7) by targeted mutagenesis (such as gene disruption, replacement of a wild-type gene allele with a variant allele encoding one or more hexose transporters with decreased function, activity and/or stability and the like). Alternatively, conventional chemically induced mutagenesis can also be used. In certain other embodiments, rather than engineering the hexose transporters, the regulatory networks controlling the expression of one or more hexose transporters are manipulated to decrease the transcription and/or translation of the hexose transporter genes. Thus, in certain other embodiments, manipulation of genes involved in post-translational regulation of glucose transporter activity (e.g., by phosphorylation or binding of other proteins) is also considered to be within the scope of current disclosure.

As used herein, a yeast gene named “HXK1” (SEQ ID NO: 33) encodes a “hexose kinase 1 protein” (HXK1; SEQ ID NO: 34), a yeast gene named “HXK2” (SEQ ID NO: 35) encodes a “hexose kinase 2 protein” (HXK2; SEQ ID NO: 36) and a yeast gene named “GLK1” (SEQ ID NO: 37) encodes a “glucokinase 1 protein” (GLK1; SEQ ID NO: 38).

In certain embodiments, a yeast HXK1 gene comprises about 85% to 99% sequence identity to SEQ ID NO: 33 and encodes a functional HXK1 protein; a yeast HXK2 gene comprises about 85% to 99% sequence identity to SEQ ID NO: 35 and encodes a functional HXK2 protein; and a yeast GLK1 gene comprises about 85% to 99% sequence identity to SEQ ID NO: 37 and encodes a functional GLK1 protein.

As used herein, a “functional” hexose kinase 1 protein (HXK1), a “functional” hexose kinase 2 protein” (HXK2) and/or a “functional” glucokinase 1 protein (GLK1) refers to proteins (i.e., enzymes) comprising Enzyme Commission No. 2.7.1.1 (EC 2.7.1.1) activity. For example, a functional HXK1 protein, a functional HXK2 and/or a functional GLK1 are enzymes capable of phosphorylating hexose sugars (hexose+ATP) to form hexose-phosphate sugars (hexose-phosphate+ADP).

As used herein, a hexose kinase (e.g., HXK1, HXK2 and/or GLK1) protein comprising “reduced (decreased) hexose kinase activity” or “reduced (decreased) hexose kinase function” may be used interchangeably, and refer to a hexose kinase protein of the disclosure comprising a reduced (decreased) hexose kinase activity (i.e., EC 2.7.1.1 activity described above).

In the event one or more of the above referenced yeast genes (e.g., hexose transporter genes (HXTn), hexose kinase genes (HXKn), glucokinase genes (GLKn) and the like) and/or their encoded proteins have been described in the art using a different nomenclature or gene naming convention, one skilled in the art may refer to the industry standard database of yeast genes (www.yeastgenome.org) using one or more of the yeast gene/protein/primer sequence identifiers (e.g., SEQ ID NO: 1-38) disclosed herein to identify such genes and proteins of the disclosure.

The engineering of a strain can be done by a variety of methods known to those skilled in the art of microbial strain engineering/construction. These methods include, but are not limited to, inactivation of the genes encoding the hexose kinases (e.g., HXK1, HXK2, GLK1) by targeted mutagenesis (such as gene disruption, replacement of a wild-type gene allele with a variant allele encoding one or more hexose transporters with decreased function, activity and/or stability and the like). Alternatively, conventional chemically induced mutagenesis can also be used. In certain other embodiments, rather than engineering the hexose kinases, the regulatory networks controlling the expression of one or more hexose kinases are manipulated to decrease the transcription and/or translation of the hexose kinase genes. Thus, in certain other embodiments, manipulation of genes involved in post-translational regulation of hexose/glucose kinase activity is also considered to be within the scope of current disclosure.

As used herein, the phrase “fermentation stress” refers to fermentation conditions which are stressful to a yeast cell (e.g., as experienced by yeast cells during SSF of sugars to alcohol).

In certain embodiments, the phrase fermentation stress refers to a fermentation temperature above about 32° C. In other embodiments, the phrase fermentation stress refers to a fermentation temperature above about 33° C. In other embodiments, the phrase fermentation stress refers to a fermentation temperature above about 34° C. In other embodiments, the phrase fermentation stress refers to a fermentation temperature above about 35° C. or higher.

In other embodiments, the phrase fermentation stress refers to a fermentation process or condition comprising a liquefact having a high dry solids content. Thus, in certain embodiments, the phrase fermentation stress refers to a liquefact comprising a dry solids (DS) content of about 32%. In other embodiments, the phrase fermentation stress refers to a liquefact comprising a DS content of about 33%, a DS content of about 34%, or higher.

In other embodiments, phrase fermentation stress refers to a combination of high temperature and high dry solids content. For example, many wild-type strains, when placed under fermentation stress (e.g., high temperature and/or high DS content) lose the ability to finish (complete) fermentation within a fermentation time typical in grain ethanol industry (e.g., 55-70 hours). This loss of ethanologen performance is manifested by increased residual glucose content and a decrease in ethanol titer at the end of fermentation.

As used herein, the phrases “improved stress tolerance” or “enhanced stress tolerance” may be used interchangeably, and refer to a modified yeast cell of the disclosure capable finishing (completing) fermentation of sugars to ethanol under a fermentation stress condition relative to the unmodified parental (yeast) cell. In certain embodiments, a modified yeast strain comprising an improved stress tolerance is capable finishing (completing) fermentation of sugars to ethanol during a fermentation time of about 55 hours to 70 hours, relative to an unmodified (e.g., wild-type) yeast strain fermented under the same (fermentation) stress condition(s).

As used herein, the phrase “improved finishing ability” is a relative term referring to the ability of a modified yeast cell strain to finish the fermentation of sugars (e.g., glucose) to ethanol, relative to an unmodified yeast strain fermented under the same conditions. In certain embodiments, a modified yeast strain of the disclosure comprising an improved finishing ability is capable of finishing the fermentation of sugars to ethanol in seventy (70) hours or less, relative to the unmodified yeast strain. In other embodiments, a modified yeast strain of the disclosure comprising an improved finishing ability is capable of finishing the fermentation of sugars to ethanol in 69 hours, 68 hours, 67 hours, 66 hours, 65 hours, 64, hours, 63 hours, 62 hours, 61 hours, 60 hours, 59 hours, 58 hours, 57 hours, 56 hours, 55 hours or less, relative to the unmodified yeast strain.

As used herein, “yeast cells”, “yeast strains”, or simply “yeast” refer to organisms from the phyla Ascomycota and Basidiomycota. Exemplary yeast is budding yeast from the order Saccharomycetales. Particular examples of yeast are Saccharomyces spp., including but not limited to S. cerevisiae. Yeast include organisms used for the production of fuel alcohol as well as organisms used for the production of potable alcohol, including specialty and proprietary yeast strains used to make distinctive-tasting beers, wines, and other fermented beverages.

As used herein, the phrase “variant yeast cells,” “modified yeast cells,” or similar phrases refer to yeast that include genetic modifications and characteristics described herein. Variant/modified yeast do not include naturally occurring yeast.

As used herein, the terms “wild-type” and “native” are used interchangeably and refer to genes proteins or strains found in nature.

As used herein, the phrase “substantially free of an activity,” or similar phrases, means that a specified activity is either undetectable in an admixture or present in an amount that would not interfere with the intended purpose of the admixture.

As used herein, the terms “polypeptide” and “protein” (and their respective plural forms) are used interchangeably to refer to polymers of any length comprising amino acid residues linked by peptide bonds. The conventional one-letter or three-letter codes for amino acid residues are used herein and all sequence are presented from an N-terminal to C-terminal direction. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, functionally and/or structurally similar proteins are considered to be “related proteins.” Such proteins can be derived from organisms of different genera and/or species, or even different classes of organisms (e.g., bacteria and fungi). Related proteins also encompass homologs determined by primary sequence analysis, determined by secondary or tertiary structure analysis, or determined by immunological cross-reactivity.

As used herein, the term “homologous protein” refers to a protein that has similar activity and/or structure to a reference protein. It is not intended that homologs necessarily be evolutionarily related. Thus, it is intended that the term encompass the same, similar, or corresponding enzyme(s) (i.e., in terms of structure and function) obtained from different organisms. In some embodiments, it is desirable to identify a homolog that has a quaternary, tertiary and/or primary structure similar to the reference protein. In some embodiments, homologous proteins induce similar immunological response(s) as a reference protein. In some embodiments, homologous proteins are engineered to produce enzymes with desired activity(ies).

The degree of homology between sequences can be determined using any suitable method known in the art (see, e.g., Smith and Waterman, 1981; Needleman and Wunsch, 1970; Pearson and Lipman, 1988); programs such as GAP, BESTFIT, FASTA, and TFASTA in the Wisconsin Genetics Software Package (Genetics Computer Group, Madison, Wis.); and Devereux et al., 1984).

For example, PILEUP is a useful program to determine sequence homology levels. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle, (Feng and Doolittle, 1987). The method is similar to that described by Higgins and Sharp (1989). Useful PILEUP parameters including a default gap weight of 3.00, a default gap length weight of 0.10, and weighted end gaps. Another example of a useful algorithm is the BLAST algorithm, described by Altschul et al. (1990) and Karlin et al. (1993). One particularly useful BLAST program is the WU-BLAST-2 program (see, e.g., Altschul et al., 1996). Parameters “W,” “T,” and “X” determine the sensitivity and speed of the alignment. The BLAST program uses as defaults a word-length (W) of 11, the BLOSUM62 scoring matrix (see, e.g., Henikoff and Henikoff, 1989) alignments (B) of 50, expectation (E) of 10, M'S, N′-4, and a comparison of both strands.

As used herein, the phrases “substantially similar” and “substantially identical,” in the context of at least two nucleic acids or polypeptides, typically means that a polynucleotide or polypeptide comprises a sequence that has at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or even at least about 99% identity, or more, compared to the reference (i.e., wild-type) sequence. Percent sequence identity is calculated using CLUSTAL W algorithm with default parameters (e.g., see Thompson et al., 1994). Default parameters for the CLUSTAL W algorithm are:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF

Another indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

As used herein, the term “protein of interest” refers to a polypeptide that is desired to be expressed in modified yeast. Such a protein can be an enzyme, a substrate-binding protein, a surface-active protein, a structural protein, a selectable marker, or the like, and can be expressed at high levels. The protein of interest is encoded by a modified endogenous gene or a heterologous gene (i.e., gene of interest”) relative to the parental strain. The protein of interest can be expressed intracellularly or as a secreted protein.

As used herein, “deletion of a gene,” refers to its removal from the genome of a host cell. Where a gene includes control elements (e.g., enhancer elements) that are not located immediately adjacent to the coding sequence of a gene, deletion of a gene refers to the deletion of the coding sequence, and optionally adjacent enhancer elements, including but not limited to, for example, promoter and/or terminator sequences, but does not require the deletion of non-adjacent control elements.

As used herein, “disruption of a gene” refers broadly to any genetic or chemical manipulation, i.e., mutation of the hosts' DNA, that substantially prevents a cell from producing a functional gene product, e.g., a protein, in a host cell. Exemplary methods of disruption include complete or partial deletion of any portion of a gene, including a polypeptide-coding sequence, a promoter, an enhancer, or another regulatory element, or mutagenesis of the same, where mutagenesis encompasses substitutions, insertions, deletions, inversions, and combinations and variations, thereof, any of which mutations substantially prevent the production of a function gene product. A gene can also be disrupted or down-regulated using RNAi, antisense, or any other method that abolishes or attenuates gene expression. A gene can be disrupted by deletion or genetic manipulation of non-adjacent control elements.

As used herein, the terms “genetic manipulation” and “genetic alteration” are used interchangeably and refer to the alteration/change of a nucleic acid sequence. The alteration can include but is not limited to a substitution, deletion, insertion or chemical modification of at least one nucleic acid in the nucleic acid sequence.

As used herein, a “primarily genetic determinant” refers to a gene, or genetic manipulation thereof, that is necessary and sufficient to confer a specified phenotype in the absence of other genes, or genetic manipulations, thereof. However, that a particular gene is necessary and sufficient to confer a specified phenotype does not exclude the possibility that additional effects to the phenotype can be achieved by further genetic manipulations.

As used herein, a “functional polypeptide/protein” is a protein that possesses an activity, such as an enzymatic activity, a binding activity, a surface-active property, or the like, and which has not been mutagenized, truncated, or otherwise modified to abolish or reduce that activity. Functional polypeptides can be thermostable or thermolabile, as specified.

As used herein, “a functional gene” is a gene capable of being used by cellular components to produce an active gene product, typically a protein. Functional genes are the antithesis of disrupted genes, which are modified such that they cannot be used by cellular components to produce an active gene product, or have a reduced ability to be used by cellular components to produce an active gene product.

As used herein, yeast cells have been “modified to prevent the production of a specified protein” if they have been genetically or chemically altered to prevent the production of a functional protein/polypeptide that exhibits an activity characteristic of the wild-type protein. Such modifications include, but are not limited to, deletion or disruption of the gene encoding the protein (as described, herein), modification of the gene such that the encoded polypeptide lacks the aforementioned activity, modification of the gene to affect post-translational processing or stability, and combinations, thereof.

As used herein, “aerobic fermentation” refers to growth in the presence of oxygen.

As used herein, “anaerobic fermentation” refers to growth in the absence of oxygen.

The following abbreviations/acronyms have the following meanings unless otherwise specified:
° C. degrees Centigrade
bp base pairs
DNA deoxyribonucleic acid
ds or DS dry solids
EtOH ethanol
g or gm gram
g/L grams per liter
H2O water
hr or h hour
kg kilogram
M molar
mg milligram
mL or ml milliliter
ml/min milliliter per minute
mM millimolar
N normal
nm nanometer
PCR polymerase chain reaction
ppm parts per million
Δ relating to a deletion
μg microgram
μL and μl microliter
μM micromolar

### II. Modified Yeast Strains Comprising Enhanced Stress Tolerance Phenotypes

Industrial yeast stains used for ethanol production operate under highly stressful conditions, and as such, improved (enhanced) stress tolerance is one of the most desirable characteristics of such strains. In particular, among the multiple forms of stress yeast experience during simultaneous saccharification and fermentation (SSF) processes, high ethanol concentrations, high dry solids content present in liquefact, and elevated temperature are considered to be the most important factors of stress. For example, the term “ethanol tolerance” can be understood differently, depending on context. In academic literature, it often means the ability to grow in a culture media supplemented with exogenous ethanol. Such “ethanol tolerance” is not synonymous with the ability to finish (complete) the conversion of high concentrations of carbohydrate material into ethanol, the property most important for an industrial ethanologen yeast strain. Furthermore, the final concentration of ethanol in the SSF process is dependent on dry solids (DS) concentration of the raw material for the process (e.g., corn liquefact).

Therefore, from the industrial ethanol process point of view “ethanol tolerance” is largely synonymous with the ability to finish fermentation of high DS liquefact (i.e., consume all available carbohydrate and convert it into ethanol). Another important property of industrial ethanologen yeast is the fermentation rate. For example, some strains may be able to finish fermentation of the substrate (e.g., corn liquefact) and even show an improved ethanol yield, but the yield benefit comes at the expense of a longer fermentation time.

For example, fermentation of glucose by yeast begins with its reversible uptake mediated by a large array of hexose transporter (HXT) proteins. As appreciated by one skilled in the art, the glucose uptake system of yeast is quite complex. There are at least eighteen (18) hexose transporter (HXT) genes in Saccharomyces cerevisiae (i.e., HXT1-HXT17 and GAL2) (Kruckeberg, 1996), with the transporters encoded by HXT1-HXT7 genes considered to be responsible for the bulk of glucose transport activity. Likewise, different glucose transporters have significantly different kinetic properties (typically high Kₘ value correlates with high Vₘₐₓ) (Maier et al, 2002). The picture if further complicated by differences in expression rates, degradation rates, regulation patterns, and the like, of such glucose transporter proteins. The glucose uptake step is followed by an essentially irreversible step of glucose phosphorylation by ATP, which is catalyzed by one of the three kinases encoded by GLK1, HXK1 and HXK2. The hexose kinase encoded by HXK2 is responsible for most of the glucokinase activity (Walsh et al., 1991).

Previously, a number of studies were dedicated to exploring the possibility of improving yeast strains by increasing the glucose uptake rate. For example, European Patent No. EP0785275 describes yeast strains constitutively expressing hexose transporters HXT1, HXT2, HXT3, HXT4, HXT5, HXT6 or HXT7. As described in EP0785275, these yeast strains were suggested to have improved CO₂ production rates and have an advantage in both dough and ethanol production applications. Similarly, U.S. Pat. No. 6,159,725 describes an improved CO₂ production rate in baker's yeast transformed with genes of hexose transporters (HXT1, HXT2, HXT3, HXT4, HXT5, HXT6 or HXT7) under control of constitutive promoters. Rossi et al. (2010) describe a yeast strain over-expressing HXT1 and HXT7 genes which was reported to produce both ethanol and lactic acid at enhanced rates. In addition, PCT Publication No. WO2019/046043 describes an improved ethanol yield under corn ethanol process conditions by yeast strains expressing plant-derived ATP-dependent glucose transporters.

In contrast to the references set forth and described above, in an effort to improve the stress tolerance yeast ethanologen strains experience during such simultaneous saccharification and fermentation (SSF) processes, Applicant investigated the effects of limiting the glucose influx into yeast metabolism under industrially meaningful conditions. More particularly, Applicant tested the effects of such modifications on yeast performance under stressful fermentation conditions, including elevated temperatures and high dry solids concentrations (e.g., see Examples 1-3). Thus, to explore the effects of limiting the glucose influx into such yeast strains and improving stress tolerance thereof, Applicant targeted the first two steps in glucose assimilation by yeast, the transport of glucose into the cell and the initial glucose-phosphorylation step.

For example, as generally set forth below in Example 1, Applicant constructed genetically modified yeast strains derived from a parental industrial ethanologen yeast strain (FermaxGold). More particularly, the deletions/disruptions described in Examples 1-3 were introduced into the FermaxGold strain to generate modified (daughter) strains (cells) thereof (e.g., see TABLE 3). Thus, as presented in TABLE 3, modified yeast strains (cells) were constructed comprising disruptions of: (a) a single HXT1 allele (e.g., single-allele disruption; strain FGH1), (b) both HXT1 alleles (e.g., double-allele disruption; strain FGH11), (c) a single HXT1 allele and a single HXT3 allele (e.g., strain FGH1-3), (d) a single HXT1 allele and a single HXT4 allele (e.g., strain FGH1-4), (e) a single HXT3 allele (e.g., single-allele disruption; strain FGH3), (f) a single HXT4 allele (e.g., single-allele disruption; strain FGH4), (g) both HXT4 alleles (e.g., double-allele disruption; strain FGH44) and (h) disruptions of a single HXK2 allele (e.g., single-allele disruption; strain FGHK2).

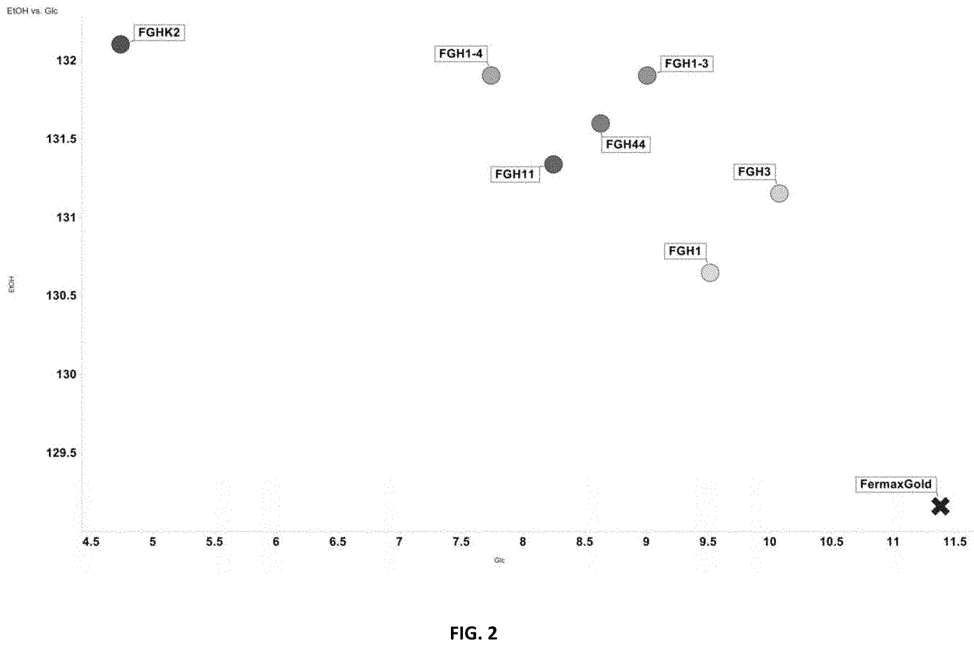

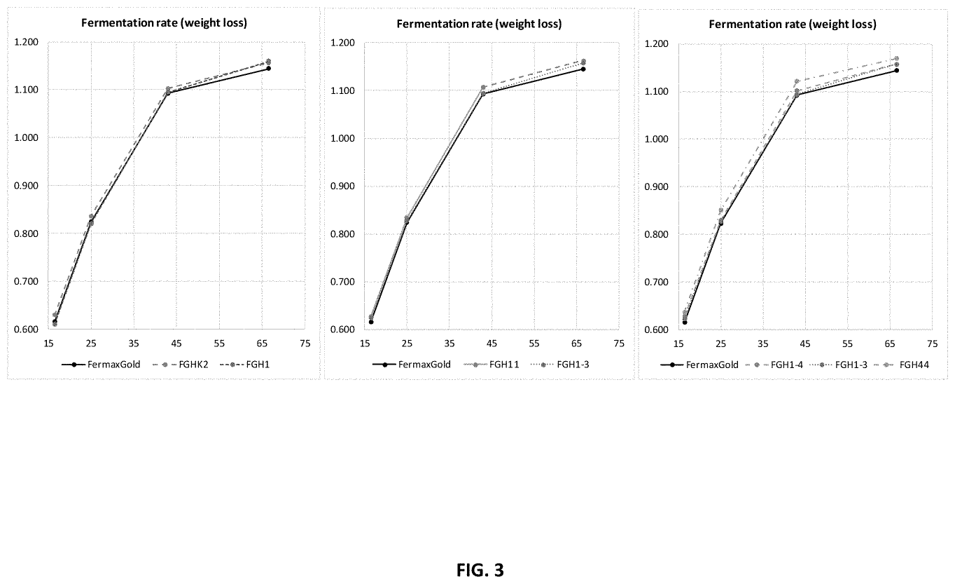

As described below in Example 2, the yeast strains constructed in Example 1 (FermaxGold, FGH1, FGH1-3, FGH1-4, FGH3, FGH4, FGH44 and FGHK2) were tested in a small scale simultaneous saccharification and fermentation (SSF) experiment designed to simulate SSF in industrial grain ethanol process, wherein fermentation was allowed to proceed at constant high temperature of 35° C. for about 66 hours. As presented in FIG. 2, which shows the effect of various gene disruptions on the efficiency of SSF at the elevated 35° C. temperature, it was surprisingly observed that all the modified strains tested in this experiment demonstrated improved stress tolerance (e.g., improved thermotolerance) relative to the wild-type parent strain (FermaxGold). More particularly, a rather consistent trend was observed, wherein single-allele deletions of HXT1 (strain FGH1), HXT3 (strain FGH3) and HXT4 (strain FGH4) result in a moderate thermotolerance enhancement, while strains carrying two disrupted alleles of HXTn genes (i.e., either a double-allele deletion of a single gene, or a combination of two single-allele deletions in two different genes) demonstrated stronger thermotolerance (FIG. 2). The strongest effect on thermotolerance was observed with a single-chromosome deletion of the hexokinase HXK2 gene (strain FGHK2). This is likely explained for the reason that the HXK2 gene is responsible for most hexokinase activity in S. cerevisiae, whereas at least seven (7) HXTn genes contribute substantially to glucose transport into the cell. Importantly, the improved glucose utilization and enhanced ethanol yields observed for the modified strains do not come at the expense of slower fermentation rate. For example, as shown in FIG. 3, all of the modified strains have fermentation rates higher than the wild-type control (FermaxGold).

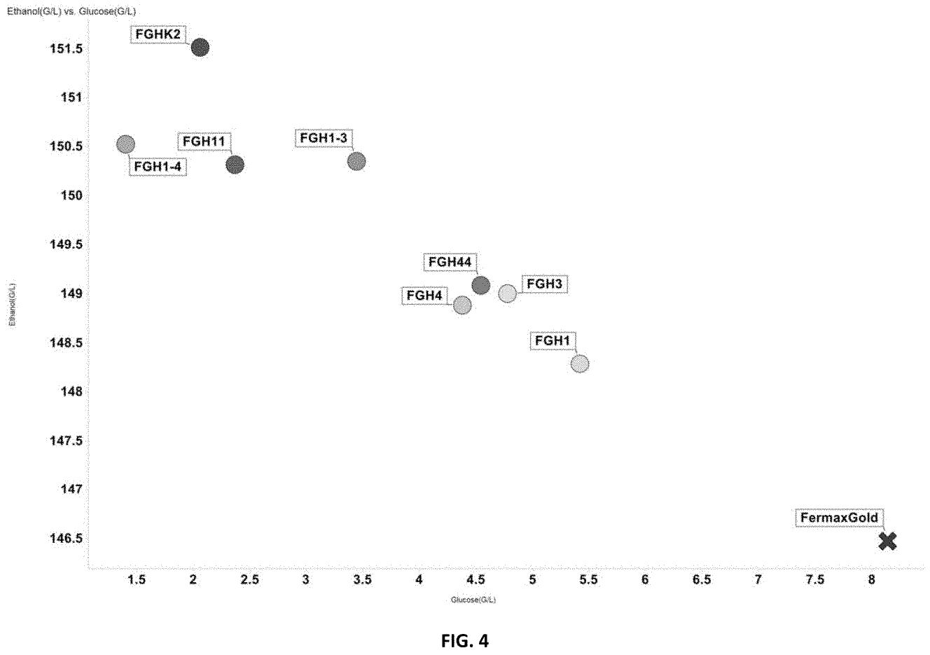

In addition, the yeast strains constructed in Example 1 (FermaxGold, FGH1, FGH1-3, FGH1-4, FGH3, FGH4, FGH44 and FGHK2) were tested in a small scale SSF experiment using very high dry solids liquefact (36% DS). For example, as described in Example 3 and presented in FIG. 4, the wild-type ethanologen yeast strain (FermaxGold) does not finish fermentation of this liquefact at sixty-five (65) hours, leaving about eight (8) grams of unfermented glucose. In contrast, it was surprisingly observed herein that all of the modified strains tested in the Example performed better than wild-type strain from which they were derived (FIG. 4). Thus, most of the modified strains follow the same trend that was observed in experiment testing the thermotolerance (Example 2), wherein a single-allele (heterozygous) deletion in one of the HXT1, HXT3 or HXT4 genes result in a moderate improvement in glucose consumption and ethanol production. Likewise, two-allele deletions, either as a homozygous deletion in one of the HXTn genes, or a combination of two single-allele deletions in two different HXTn genes tend to lead to a further improvement in glucose consumption and ethanol production. Similar to the experiment of Example 2, strain FGHK2 (carrying a single-allele deletion in HXK2 gene) demonstrates the best performance of all modified strains tested.

### III. Yeast Cells Suitable for Modification

Yeasts are unicellular eukaryotic microorganisms classified as members of the fungus kingdom and include organisms from the phyla Ascomycota and Basidiomycota. Yeast that can be used for alcohol production include, but are not limited to, Saccharomyces spp., including S. cerevisiae, as well as Kluyveromyces, Lachancea and Schizosaccharomyces spp. Numerous yeast strains are commercially available, many of which have been selected or genetically engineered for desired characteristics, such as high alcohol production, rapid growth rate, and the like. Some yeasts have been genetically engineered to produce heterologous enzymes, such as glucoamylase or α-amylase.

### IV. Use of Modified Yeast for Increased Alcohol Production

Certain embodiments of the disclosure are related to modified yeast strains and methods thereof for increasing the efficiency of alcohol production using such modified yeast in fermentation reactions/processes. The methods include performing fermentation at an elevated temperature and/or at increased liquefact dry solids (DS) concentrations, optionally, for a shorter period of time, compared to an otherwise equivalent fermentation performed using the parental cells.

For example, fermentation using the modified yeast cells may be performed at 1° C., 2° C., 3° C., 4° C., 5° C., 6° C., 7° C., 8° C., 9° C., or even 10° C., above the temperature used for fermentation with the parental yeast cells, provided that the modified yeast is capable of making at least the same amount of alcohol at the increased temperature as the parental yeast make at the reference temperature.

Likewise, the fermentation using the modified yeast cells may be performed with a 1% increase in liquefact DS content, a 2% increase in liquefact DS content, a 3% increase in liquefact DS content, or even a 5% increase in liquefact DS content, above the liquefact DS content used for the fermentation with the parental yeast cells, provided that the modified yeast is capable of making at least the same amount of alcohol at the increased liquefact DS content as the parental yeast make at the reference liquefact DS content.

The higher temperature fermentation and/or increased fermentation liquefact DS content may optionally be run for 99%, 98%, 97%, 96%, 95%, 94%, 93%, 92%, 91, 90%, 85%, 80%, 75%, 70% or less, compared to the amount of time required for fermentation using the parental yeast, provided that the modified yeast is capable of making at least the same amount of alcohol at the increased temperature and/or increased liquefact DS content as the parental yeast make at the reference temperature and time and/or at the reference liquefact DS content and time.

Alternatively, the methods include performing fermentation at about the same temperature and about the same length of time compared to an otherwise equivalent fermentation performed using the parental cells, wherein the modified yeast cells produce at least 1%, at least 2%, at least 3%, at least 4%, or even at least 5% more alcohol than the parental yeast under equivalent conditions.

The advantages of the modified yeast in terms of performing fermentations at increased temperatures, performing fermentations at increased liquefact DS content, performing fermentations for shorter period of time, and increasing alcohol yield under conventional fermentation conditions, can be combined to maximize benefit to a particular alcohol production facility.

In some embodiments, in situ production removal (ISPR) may be utilized to remove product alcohol from fermentation as the product alcohol is produced by the microorganism. Processes for removing solids and producing and recovering alcohols from fermentation broth are described in U.S. Patent Application No. 2014/0073820 and U.S. Patent Application No. 2015/0267225.

Alcohol production from a number of carbohydrate substrates, including but not limited to corn starch, sugar cane, cassava, and molasses, is well known, as are innumerable variations and improvements to enzymatic and chemical conditions and mechanical processes. The present compositions and methods are believed to be fully compatible with such substrates and conditions.

### VI. Molecular Biology

As generally set forth above, certain embodiments of the disclosure are related to compositions and methods for increasing the efficiency of alcohol production using modified yeast cells of the disclosure in fermentation reactions/processes. For example, in certain embodiments a modified yeast strain is derived from parental yeast strain, wherein the modified strain comprises a reduced (attenuated) ability to take up (transport) glucose compared to the parental strain when fermented under conditions for the production of ethanol and/or the modified strain comprises a reduced (attenuated) ability to catalyze the phosphorylation of glucose into glucose-phosphate compared to the parental cells when fermented under conditions for the production of ethanol. Thus, in certain embodiments, the disclosure is related to modified yeast strains comprising an enhanced stress tolerance phenotype compared to the parental cells when fermented under conditions for the production of ethanol.

In certain embodiments, a modified yeast strain comprising an enhanced stress tolerance phenotype of the disclosure (i.e., relative to the parental strain) comprises the ability to ferment glucose to ethanol at elevated temperatures, the ability to ferment glucose to ethanol at a high liquefact DS content, the ability to ferment glucose to ethanol at an increased rate of ethanol production and the like.

More particularly, in certain embodiments, a modified yeast strain of the disclosure comprising an enhanced stress tolerance phenotype comprises a genetic alteration (modification) of one or more genes selected from the group consisting of a HXT1 gene, a HXT2 gene, a HXT3 gene, a HXT4 gene, a HXT5 gene, a HXT6 gene, a HXT7 gene, a HXK1 gene, a HXK2 gene and a GLK1 gene. Thus, certain embodiments of the disclosure provide compositions and methods for genetically modifying (altering) a parental yeast strain (cell) of the disclosure to generate modified yeast strain (cell) thereof.

Methods and compositions for genetically modifying yeast cells, include, but are not limited to, (a) the introduction, substitution, or removal of one or more nucleotides in a gene of the disclosure (or an ORF thereof), or the introduction, substitution, or removal of one or more nucleotides in a regulatory element required for the transcription or translation of the gene or ORF thereof, (b) gene disruption, (c) gene conversion, (d) gene deletion, (e) gene down-regulation, (f) site specific mutagenesis and/or (g) random mutagenesis.

In certain embodiments, a modified yeast cell of the disclosure is constructed by reducing or eliminating the expression of a HXT1 gene, a HXT2 gene, a HXT3 gene, a HXT4 gene, a HXT5 gene, a HXT6 gene, a HXT7 gene, a HXK1 gene, a HXK2 gene and/or a GLK1 gene, using methods well known in the art, for example, insertions, disruptions, replacements, or deletions. The portion of the gene to be modified or inactivated may be, for example, the coding region or a regulatory element required for expression of the coding region.

An example of such a regulatory or control sequence may be a promoter sequence or a functional part thereof, (i.e., a part which is sufficient for affecting expression of the nucleic acid sequence). Other control sequences for modification include, but are not limited to, a leader sequence, a propeptide sequence, a signal sequence, a transcription terminator, a transcriptional activator and the like.

In certain other embodiments a modified yeast cell is constructed by gene deletion to eliminate or reduce the expression of at least one of the aforementioned genes of the disclosure. Gene deletion techniques enable the partial or complete removal of the gene(s), thereby eliminating their expression, or expressing a non-functional (or reduced activity) protein product. In such methods, the deletion of the gene(s) may be accomplished by homologous recombination using a plasmid that has been constructed to contiguously contain the 5′ and 3′ regions flanking the gene. The contiguous 5′ and 3′ regions may be introduced into a yeast cell, for example, on a temperature-sensitive plasmid, in association with a second selectable marker at a permissive temperature to allow the plasmid to become established in the cell. The cell is then shifted to a non-permissive temperature to select for cells that have the plasmid integrated into the chromosome at one of the homologous flanking regions. Selection for integration of the plasmid is effected by selection for the second selectable marker. After integration, a recombination event at the second homologous flanking region is stimulated by shifting the cells to the permissive temperature for several generations without selection. The cells are plated to obtain single colonies and the colonies are examined for loss of both selectable markers (see, e.g., Perego, 1993). Thus, a person of skill in the art may readily identify nucleotide regions in the gene's coding sequence and/or the gene's non-coding sequence suitable for complete or partial deletion.

In other embodiments, a modified yeast cell of the disclosure is constructed by introducing, substituting, or removing one or more nucleotides in the gene or a regulatory element required for the transcription or translation thereof. For example, nucleotides may be inserted or removed so as to result in the introduction of a stop codon, the removal of the start codon, or a frame-shift of the open reading frame. Such a modification may be accomplished by site-directed mutagenesis or PCR generated mutagenesis in accordance with methods known in the art. Thus, in certain embodiments, a gene of the disclosure is inactivated by complete or partial deletion.

In other embodiments a modified yeast cell is constructed by the process of gene conversion (e.g., see Iglesias and Trautner, 1983). For example, in the gene conversion method, a nucleic acid sequence corresponding to the gene(s) is mutagenized in vitro to produce a defective nucleic acid sequence, which is then transformed into the parental cell to produce a defective gene. By homologous recombination, the defective nucleic acid sequence replaces the endogenous gene. It may be desirable that the defective gene or gene fragment also encodes a marker which may be used for selection of transformants containing the defective gene. For example, the defective gene may be introduced on a non-replicating or temperature-sensitive plasmid in association with a selectable marker. Selection for integration of the plasmid is effected by selection for the marker under conditions not permitting plasmid replication. Selection for a second recombination event leading to gene replacement is effected by examination of colonies for loss of the selectable marker and acquisition of the mutated gene. Alternatively, the defective nucleic acid sequence may contain an insertion, substitution, or deletion of one or more nucleotides of the gene, as described below.

In other embodiments, a modified yeast cell is constructed by established anti-sense techniques using a nucleotide sequence complementary to the nucleic acid sequence of the gene (Parish and Stoker, 1997). More specifically, expression of the gene by a yeast cell may be reduced (down-regulated) or eliminated by introducing a nucleotide sequence complementary to the nucleic acid sequence of the gene, which may be transcribed in the cell and is capable of hybridizing to the mRNA produced in the cell. Under conditions allowing the complementary anti-sense nucleotide sequence to hybridize to the mRNA, the amount of protein translated is thus reduced or eliminated. Such anti-sense methods include, but are not limited to RNA interference (RNAi), small interfering RNA (siRNA), microRNA (miRNA), antisense oligonucleotides, and the like, all of which are well known to the skilled artisan.

In other embodiments, a modified yeast cell is produced/constructed via CRISPR-Cas9 editing. For example, a gene encoding a protein of interest can be edited or disrupted (or deleted or down-regulated) by means of nucleic acid guided endonucleases, that find their target DNA by binding either a guide RNA (e.g., Cas9) and Cpf1 or a guide DNA (e.g., NgAgo), which recruits the endonuclease to the target sequence on the DNA, wherein the endonuclease can generate a single or double stranded break in the DNA. This targeted DNA break becomes a substrate for DNA repair, and can recombine with a provided editing template to disrupt or delete the gene. For example, the gene encoding the nucleic acid guided endonuclease (for this purpose Cas9 from S. pyogenes) or a codon optimized gene encoding the Cas9 nuclease is operably linked to a promoter active in the yeast cell and a terminator active in yeast cell, thereby creating a yeast Cas9 expression cassette. Likewise, one or more target sites unique to the gene of interest are readily identified by a person skilled in the art. For example, to build a DNA construct encoding a gRNA-directed to a target site within the gene of interest, the variable targeting domain (VT) will comprise nucleotides of the target site which are 5′ of the (PAM) proto-spacer adjacent motif (TGG), which nucleotides are fused to DNA encoding the Cas9 endonuclease recognition domain for S. pyogenes Cas9 (CER). The combination of the DNA encoding a VT domain and the DNA encoding the CER domain thereby generate a DNA encoding a gRNA. Thus, a yeast cell expression cassette for the gRNA is created by operably linking the DNA encoding the gRNA to a promoter active in yeast cells and a terminator active in yeast cells.

In certain embodiments, the DNA break induced by the endonuclease is repaired/replaced with an incoming sequence. For example, to precisely repair the DNA break generated by the Cas9 expression cassette and the gRNA expression cassette described above, a nucleotide editing template is provided, such that the DNA repair machinery of the cell can utilize the editing template. For example, about 500 bp 5′ of targeted gene can be fused to about 500 bp 3′ of the targeted gene to generate an editing template, which template is used by the yeast host's machinery to repair the DNA break generated by the RGEN.

The Cas9 expression cassette, the gRNA expression cassette and the editing template can be co-delivered to filamentous fungal cells using many different methods (e.g., protoplast fusion, electroporation, natural competence, or induced competence). The transformed cells are screened by PCR amplifying the target gene locus, by amplifying the locus with a forward and reverse primer. These primers can amplify the wild-type locus or the modified locus that has been edited by the RGEN. These fragments are then sequenced using a sequencing primer to identify edited colonies.

In yet other embodiments, a modified yeast cell is constructed by random or specific mutagenesis using methods well known in the art, including, but not limited to, chemical mutagenesis (see, e.g., Hopwood, 1970) and transposition (see, e.g., Youngman et al., 1983). Modification of the gene may be performed by subjecting the parental cell to mutagenesis and screening for mutant cells in which expression of the gene has been reduced or eliminated. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods.

Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosoguanidine (NTG), O-methyl hydroxylamine, nitrous acid, ethyl methane sulphonate (EMS), sodium bisulphite, formic acid, and nucleotide analogues. When such agents are used, the mutagenesis is typically performed by incubating the parental cell to be mutagenized in the presence of the mutagenizing agent of choice under suitable conditions, and selecting for mutant cells exhibiting reduced or no expression of the gene.

### EXAMPLES

Certain aspects of the present disclosure may be further understood in light of the following examples, which should not be construed as limiting. Modifications to materials and methods will be apparent to those skilled in the art.

### Example 1

### Construction of Derivatives of an Industrial Ethanologen Strain with Deletions in Hexose Transporter and Hexokinase Genes

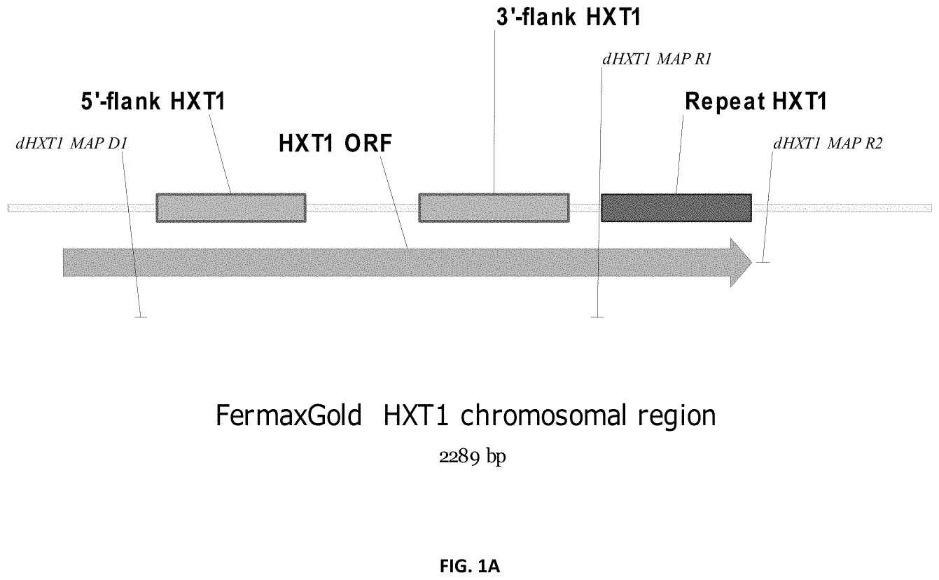

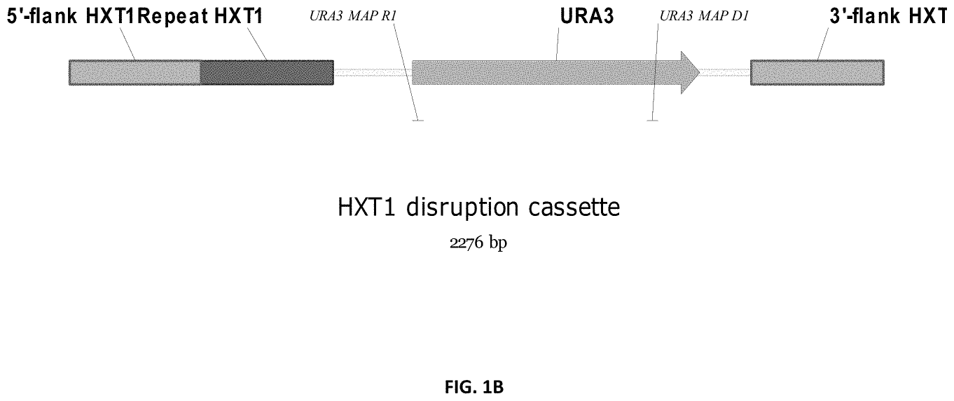

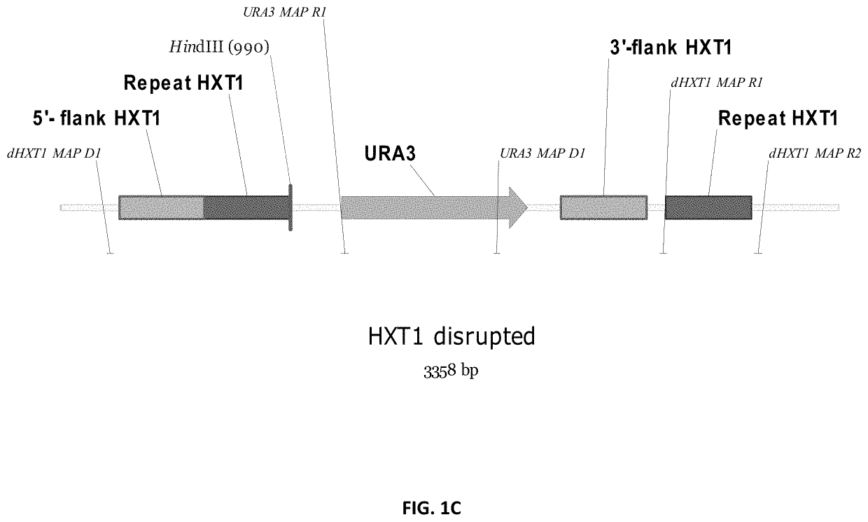

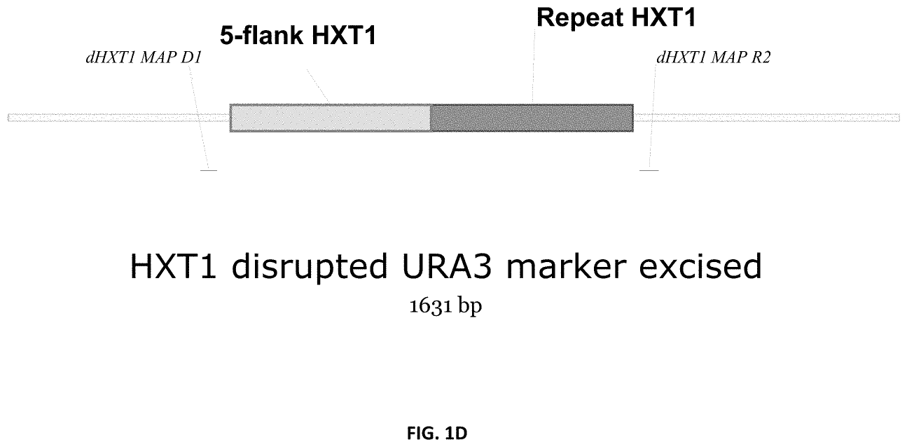

The starting point for the construction work was an industrial ethanologen yeast strain named FermaxGold, which strain comprises a deletion in URA3 gene making it a uridine auxotroph. For example, all deletions/disruptions were introduced into the FermaxGold strain following the same “gene disruption by double crossover” paradigm commonly used in yeast molecular biology studies. More particularly, DNA constructs comprising a 5′-flanking sequence, a “repeat” sequence, a URA3 gene and a 3′-flanking sequence were designed and ordered from a DNA synthesis provider. FIG. 1 illustrates an exemplary design of an HXT1 gene disruption construct used herein to assess the gene disruptions of the HXT1 gene. Likewise, the gene disruption cassettes for HXT3, HXT4 and HXK2 genes were designed and synthesized in the same way. Thus, SEQ ID NO: 1 shows the DNA sequence of the HXT1 disruption cassette, SEQ ID NO: 2 shows the DNA sequence of the HXT3 disruption cassette, SEQ ID NO: 3 shows the DNA sequence of the HXT4 disruption cassette, and SEQ ID NO: 4 shows the DNA sequence of the HXK2 disruption cassette.

After transformation of a ura3 host strain with any of the above described disruption cassettes (SEQ ID NOS: 1-4) to uridine prototrophy, correct integration of the disruption cassette in individual transformants was verified by PCR. The primers used for this purpose are listed in TABLE 1, while the primer combinations used at specific construction steps and correct PCR product sizes are listed in TABLE 2. Transformants producing PCR products of the expected size, were further purified by an additional round of sub-cloning. To excise the URA3 marker after a successful gene disruption step, the strain carrying the disrupted allele was cultivated on a standard yeast mineral medium supplemented with 1 g/l of fluoroorotic acid and 100 mg/l uridine. Again, DNA was extracted from individual transformants, used as a template for PCR and correct clones identified and purified (see, TABLE 1 and TABLE 2).

When gene disruption is carried out in a manner described in this example, only one allele of the targeted gene was disrupted in a single experiment. FermaxGold is a diploid strain, and as such, to disrupt both alleles of the same gene, two (2) rounds of the procedure outlined here have to be carried out: disruption of the first allele, marker excision, and disruption of the second allele. At this point, marker excision can be performed again and a different gene can be disrupted using exactly the same technique. In this manner, various combination of heterozygous and homozygous gene deletions can be accumulated in a single strain. TABLE 3 lists the resulting strains that were used in physiological experiments. For clarity, intermediate uridine-auxotrophic strains are not listed.

TABLE 1
PRIMERS USED TO CONFIRM CORRECT CHROMOSOMAL
MODIFICATIONS OF HXT1, HXT3 AND HXT4 GENES

| | | SEQ |
|---|---|---|
| | | ID |
| Primer name | Primer sequence | NO |
| HXT1_MAP_D1 | GGTGCCTACGTAATGGTTTCTATCTGTTGTG | 5 |
| HXT1_MAP_R1 | CAATTGGAGCCCATGTAGTGGCGAAACAAAAG | 6 |
| HXT1_MAP_R2 | CTGTATAAGTCATTAAAATATGCATATTGAGC | 7 |
| | TTG | |
| HXT3_MAP_D1 | GGGTTGCATATAAATACAGGCGCTGTTTTATC | 8 |
| HXT3_MAP_R1 | CGTTAAAAACGGTAGTACCATAGTAGAAGAAA | 9 |
| | TAG | |
| HXT3_MAP_R2 | CAAGAAACCCCACAACCAATTAGCAGCTGTAG | 10 |
| HXT4_MAP_D1 | GCTTCAACACTGGGGAATGAATAATATGTCATC | 11 |
| HXT4_MAP_R1 | GCACCCATGATCAAACGTTGGAAAACCTTAGTC | 12 |
| HXT4_MAP_R2 | CCAAACAGCCCATGAAAACGTAACCGTAGTAG | 13 |
| HXK2_MAP_D1 | GGAATATAATTCTCCACACATAATAAGTACGCT | 14 |
| HXK2_MAP_R1 | CCTTGTTTGTACATGTCCATCAAGGCCAAAC | 15 |
| HXK2_MAP_R2 | CACCAGCACCGGAACCATCTTCAGCAGGAACAA | 16 |
| | TC | |
| URA3_MAP_D1 | CCGTGGATGATGTGGTCTCTACAGGATCTGAC | 17 |
| URA3_MAP_R1 | GCAGCACGTTCCTTATATGTAGCTTTCGACATG | 18 |

TABLE 2
SIZES OF PCR FRAGMENTS USED TO CONFIRM CORRECT
INTEGRATION OF DISRUPTION CASSETTES AND
EXCISION OF THE SELECTABLE MARKER GENE

| | | | Correct PCR |
|---|---|---|---|
| | | | fragment size, |
| Sense primer | Anti-sense primer | Construction step | kb |
| HXT1_MAP_D1 | URA3_MAP_R1 | HXT1 disruption | 1.05 |
| URA3_MAP_D1 | HXT1_MAP_R1 | HXT1 disruption | 1.16 |
| HXT3_MAP_D1 | URA3_MAP_R1 | HXT3 disruption | 1.11 |
| URA3_MAP_D1 | HXT3_MAP_R1 | HXT3 disruption | 0.69 |
| HXT4_MAP_D1 | URA3_MAP_R1 | HXT4 disruption | 1.06 |
| URA3_MAP_D1 | HXT4_MAP_R1 | HXT4 disruption | 0.72 |
| HXK2_MAP_D1 | URA3_MAP_R1 | HXK2 disruption | 0.91 |
| URA3_MAP_D1 | HXK2_MAP_R1 | HXK2 disruption | 0.67 |
| HXT1_MAP_D1 | HXT1_MAP_R2 | URA3 excision at HXT1 locus | 0.84 |
| HXT3_MAP_D1 | HXT3_MAP_R2 | URA3 excision at HXT3 locus | 0.89 |
| HXT4_MAP_D1 | HXT4_MAP_R2 | URA3 excision at HXT4 locus | 0.93 |
| HXK2_MAP_D1 | HXK2_MAP_R2 | URA3 excision at HXK2 locus | 0.69 |

TABLE 3
STRAINS USED IN THIS STUDY

| Strain name | Relevant genotype | Comment |
|---|---|---|
| FermaxGold | Wild-Type | Industrial ethanologen strain (diploid) |
| FermaxGold ura3 | ura3 | Uridine auxotrophic derivative |
| | | of FermaxGold |
| FGH1 | FG ura3 ΔHXT1::URA3/ HXT1 | Single-allele deletion of HXT1 |
| FGH11 | FG ura3 ΔHXT1::URA3/Δ HXT1 | Double-allele deletion of HXT1 |
| FGH1-3 | FG ura3 ΔHXT1/HXT1 ΔHXT3::URA3/HXT3 | Single-allele deletions of HXT1 and HXT3 |
| FGH1-4 | FG ura3 ΔHXT1/HXT1 ΔHXT4::URA3/HXT4 | Single-allele deletions of HXT1 and HXT4 |
| FGH3 | FG ura3 ΔHXT3::URA3/ HXT3 | Single-allele deletion of HXT3 |
| FGH4 | FG ura3 ΔHXT4::URA3/ HXT4 | Single-allele deletion of HXT4 |
| FGH44 | FG ura3 ΔHXT4::URA3/Δ HXT4 | Double-allele deletion of HXT4 |
| FGHK2 | FG ura3 Δhxk2::URA3/ HXK2 | Single-allele deletion of HXK2 |

### Example 2

### Strains with Attenuated Glucose Uptake/Phosphorylation Capacity are More Thermostable than a Wild-Type Strain

The strains FermaxGold, FGH1, FGH1-3, FGH1-4, FGH3, FGH4, FGH44 and FGHK2 (see, TABLE 3) set forth in Example 1 were tested in a small scale simultaneous saccharification and fermentation (SSF) experiment designed to simulate SSF in industrial grain ethanol process. Liquefact (34.2% dry solids (DS) content, from Cardinal LLC ethanol plant, Union City, Ind.) was supplemented with 600 mg/l urea and 20 mg/l of purified glucoamylase CS4 (US2016/0068879) immediately before use. Ten (10) grams (+/−100 mg) of such liquefact was placed into a 20 gas chromatography vial equipped with air-tight lid. Gas outlet was provided by a 30 gauge (0.3×13 mm) hypodermic needle. The actual weight of liquefact in each vial was recorded with +/−0.1 mg accuracy. SSF was started by addition of a slurry of freshly grown yeast strains to initial ˜3×10⁶ cells per ml. The total weight of each vial at the start of SSF was recorded with +/−0.1 mg accuracy. Fermentation was allowed to proceed at constant high temperature (35° C.) for about 66 hours. Weight loss of each vial due to the production of CO₂ was followed over time. At the end of SSF, the fermented liquefact was sterile-filtered and the filtrate subjected to the analysis by HPLC. An otherwise similar control experiment was run at optimal (32° C.) temperature. In this experiment, all strains finished fermentation with less than 2 g/l residual glucose.

FIG. 2 shows the effect of various gene deletions on the efficiency of SSF at elevated (35° C.) temperature. The wild-type strain (FermaxGold), finishes fermentation with about 11.5 g/l residual glucose, while all the mutants tested in this experiment perform better. For example, a rather consistent trend is observed: single-allele deletions of HXT1 (strain FGH1), HXT3 (strain FGH3) and HXT4 (strain FGH4) result in only a moderate effect, while strains carrying two disrupted alleles of HXTn genes (i.e., either a double-allele deletion of a single gene, or a combination of two single-allele deletions in two different genes) demonstrate stronger thermotolerance.

The strongest effect on thermotolerance was observed with a single-chromosome deletion of HXK2 gene (strain FGHK2). This is likely explained for the reason that the HXK2 gene is responsible for most hexokinase activity in S. cerevisiae, while at least 7 HXTn genes contribute substantially to glucose transport into the cell. Importantly, the improved glucose utilization and enhanced ethanol yield observed with the mutant strains of the disclosure does not come at the expense of slower fermentation rate. As can be seen from the data on FIG. 3, all of the mutant strains have fermentation rate higher than the wild-type control (FermaxGold).

### Example 3

### Strains with Attenuated Glucose Uptake/Phosphorylation Capacity Perform Better in High Dry Solids Liquefact than a Wild-Type Strain

In the instant example, strains FermaxGold, FGH1, FGH1-3, FGH1-4, FGH3, FGH4, FGH44 and FGHK2 (TABLE 3) were also tested in a small scale SSF experiment using very high dry solids liquefact (36% DS). Otherwise, the experimental setup was similar to that of Example 2 (32° C. fermentation temperature, with the same urea and glucoamylase dosages). The wild-type ethanologen yeast strain (FermaxGold; FIG. 4) does not finish fermentation of this liquefact at sixty-five (65) hours, leaving about eight (8) grams of unfermented glucose.

In contrast, all tested strains attenuated in glucose uptake or phosphorylation (TABLE 3) perform better than wild-type strain from which they are derived (FIG. 4). Most strains follow the same trend that was observed in experiment testing the thermotolerance of the engineered strains of TABLE 3. A single-allele (heterozygous) deletion in one of the HXT1, HXT3 or HXT4 genes results in a moderate improvement in glucose consumption and ethanol production. Two-allele deletions either as a homozygous deletion in of the HXTn genes, or a combination of two single-allele deletions in two different HXTn genes tend to lead to a further improvement. Similar to the experiment of Example 2, strain FGHK2 (carrying a single-allele deletion in HXK2 gene) demonstrates the best performance of the whole group of strains tested.

### REFERENCES

European Patent No. EP0785275
PCT Publication No. WO2019/046043
U.S. Pat. No. 6,159,725
U.S. Patent Publication No. 2014/0073820
U.S. Patent Application No. 2015/0267225
U.S. Patent Publication No. 2016/0068879
Altschul et al., J. Mol. Biol. 215:403-410, 1990.
Altschul et al., Meth. Enzymol. 266:460-480, 1996.
Devereux et al., Nucleic Acids Res. 12:387-395, 1984.
Feng and Doolittle, J. Mol. Evol. 35:351-360, 1987.
Henikoff and Henikoff, Proc. Natl. Acad. Sci. USA 89:10915, 1989.
Higgins and Sharp, CABIOS 5:151-153, 1989.
Karlin et al., Proc. Natl. Acad. Sci. USA 90:5873-87, 1993.
Kruckeberg “The hexose transporter family of Saccharomyces cerevisiae”, Arch. Microbiol. 166(5): 283-292, 1996.
Maier et al., “Characterisation of glucose transport in Saccharomyces cerevisiae with plasma membrane vesicles (countertransport) and intact cells (initial uptake) with single Hxt1, Hxt2, Hxt3, Hxt4, Hxt6, Hxt7 or Gal2 transporters”, FEMS Yeast Research, Volume 2, Issue 4, pages 539-550, 2002.
Needleman and Wunsch, J. Mol. Biol., 48:443, 1970.
Pearson and Lipman, Proc. Natl. Acad. Sci. USA 85:2444, 1988.
Rossi et al., “Effect of HXT 1 and HXT 7 hexose transporter overexpression on wild-type and lactic acid producing Saccharomyces cerevisiae cells”, Microbial Cell Factories vol. 9, No. 15, 2010.
Smith and Waterman, Adv. Appl. Math. 2:482, 1981.
Thompson et al., Nucleic Acids Res. 22:4673-4680, 1994.
Walsh et al., “Saccharomyces cerevisiae null mutants in glucose phosphorylation: metabolism and invertase expression”, Genetics Vol. 128, No. 3, pages 521-527, 1991.

1. A modified yeast cell derived from a parental cell, the modified cell comprising an attenuated ability to transport glucose and/or an attenuated ability to catalyze the phosphorylation of glucose into glucose 6-phosphate compared to the parental cell, wherein the modified cell comprises an enhanced stress tolerance phenotype compared to the parental cell when fermented under identical conditions for the production of ethanol.

2. The modified cell of claim 1, wherein the attenuated ability to transport glucose comprises at least one genetic alteration that causes the modified cell to produce a decreased amount of a functional HXT1 polypeptide, a decreased amount of a functional HXT2 polypeptide, a decreased amount of a functional HXT3 polypeptide, a decreased amount of a functional HXT4 polypeptide, a decreased amount of a functional HXT5 polypeptide, a decreased amount of a functional HXT6 polypeptide and/or a decreased amount of a functional HXT7 polypeptide compared to the parental cell.

3. The modified cell of claim 1, wherein the attenuated ability to catalyze the phosphorylation of glucose into glucose 6-phosphate comprises a genetic alteration that causes the modified cell to produce a decreased amount of a functional HXK1 polypeptide, a decreased amount of a functional HXK2 polypeptide, and/or a decreased amount of a functional GLK1 polypeptide compared to the parental cell.

4. The modified cell of claim 1, wherein the modified cell does not produce a functional HXT1 polypeptide, a functional HXT2 polypeptide, a functional HXT3 polypeptide, a functional HXT4 polypeptide, a functional HXT5 polypeptide, a functional HXT6 polypeptide and/or a functional HXT7 polypeptide.

5. The modified cell of claim 1, wherein the modified cell does not produce a functional HXK1 polypeptide, a functional HXK2 polypeptide, and/or a functional GLK1 polypeptide.

6. The modified cell of claim 1, further comprising an exogenous gene encoding a carbohydrate processing enzyme.

7. The modified cell claim 1, wherein the enhanced stress tolerance phenotype is an enhanced ability to ferment glucose to ethanol at an elevated fermentation temperature.

8. The modified cell claim 1, wherein the enhanced stress tolerance phenotype is an enhanced ability to finish fermentation of glucose to ethanol in the presence of a high dry solids (DS) liquefact concentration.

9. The modified cell claim 1, wherein the enhanced stress tolerance phenotype is an enhanced rate of ethanol production.

10. The modified cell claim 1, wherein the enhanced stress tolerance phenotype is an increased ethanol yield.

11. A modified yeast cell derived from a parental cell, the modified cell comprising an attenuated ability to transport glucose and/or an attenuated ability to catalyze the phosphorylation of glucose into glucose 6-phosphate compared to the parental cell, wherein the modified cell produces during fermentation an increased amount of ethanol compared to parental cell when fermented under identical conditions for the production of ethanol.

12. A method for producing a modified yeast cell, the method comprising introducing a genetic alteration into a parental yeast cell, which genetic alteration reduces or prevents the production of a functional HXT1 polypeptide, a functional HXT2 polypeptide, a functional HXT3 polypeptide, a functional HXT4 polypeptide, a functional HXT5 polypeptide, a functional HXT6 polypeptide, a functional HXT7 polypeptide, a functional HXK1 polypeptide, a functional HXK2 polypeptide and/or a functional GLK1 polypeptide compared to the parental cell, thereby producing a modified cell that produces during fermentation an increased amount of ethanol compared to the parental cells under equivalent fermentation conditions.

13. A modified yeast cell produced by the method of claim 12.

14. A method for producing increased amounts of ethanol in a yeast fermentation process, the method comprising fermenting a modified cell of claim 1 or claim 11 under suitable conditions for the production of ethanol, wherein the modified strain produces an increased amount of ethanol relative to the parental strain when fermented under identical conditions.
